(19) United States
(12) Patent Application Publication
(10) Pub. No.: US 2023/0190160 A1
(43) Pub. Date: Jun. 22, 2023

(54) SYSTEM AND METHOD FOR DETERMINING INTOXICANT IMPAIRMENT

(71) Applicant: Bloonics Holding B.V., Eindhoven (NL)

(72) Inventors: Alexei Leonidovich Beresnev, Columbia, MD (US); Daniel Allen Cohen, Columbia, MD (US); Stefan Sandberg, Aelvaengen (SE)

(21) Appl. No.: 17/993,432

(22) Filed: Nov. 23, 2022

Related U.S. Application Data
(60) Provisional application No. 63/282,918, filed on Nov. 24, 2021.

(51) Int. Cl. A61B 5/16; A61B 5/00; G01N 33/543; B01L 3/00

(52) U.S. Cl. CPC A61B 5/165; A61B 5/163; A61B 5/4035; G01N 33/54366; B01L 3/502715; B01L 2200/10

(57) ABSTRACT

A system and method for collection and interpretation of data for determining impairment by intoxicant(s) in a subject at the point of collection utilizes of multiple sensors which may comprise both a live-cell assay in a disposable cartridge for determining the presence of intoxicant(s) and an eye scanner for determining vital signs and neurological state of the subject. The cartridge may be equipped to intake a sample, process it, and/or interact it with one or more eukaryotic cell-based biosensors. A cartridge may function as an optical interface to relay signals from the biosensors to a detector. The eye scanner may be equipped with optical sensors for the detecting both vital signs and neurological state. Each optical sensor may be spectrally filtered to identify a biomarker or set of biomarkers. Another method utilizes the eye scanner to train a model to identify additional biomarkers within the live-cell assay on a cartridge to increase the specificity for impairment by the primary analyte. One such example of this approach is to identify combinations of cannabinoids, both endogenous and plant-derived, which when found together with D⁹-tetrahydrocannabinol (THC), confer a higher probability that THC is actively impairing the test subject.

### CROSS REFERENCE TO RELATED APPLICATIONS

This application is a non-provisional application claiming priority from U.S. Provisional Application No. 63/282,918, entitled “System And Method For Determining Intoxicant Impairment”, and filed Nov. 24, 2021, the disclosure in which is incorporated in its entirety herein by this reference.

### TECHNICAL FIELD

The disclosure generally relates to systems and methods for the collection and interpretation of data for the determination of impairment by intoxicants in a subject at the point of collection.

### BACKGROUND OF THE DISCLOSURE

Point-of-collection testing is widely used in both law enforcement and in the workplace for the detection of drugs of impairment. The drugs of impairment paneled are often determined according to the guidance provided by The Substance Abuse and Mental Health Services Administration (SAMHSA) and consist of: Cannabis, Methamphetamines, Cocaine, Phencyclidine, and Opiates. However, these panels are not always specific for impairment at the time of collection and instead detect inactive metabolites with longer half-lives. This is not always a desirable attribute of an assay, for example in the case of cannabis where a test operator may be concerned only with active impairment by THC.

One of the most pressing questions is how to determine intoxication (motor impairment, slowed reflexes, etc.) resulting from cannabis consumption. This poses a potential safety risk both on roadways and in workplaces where heavy equipment is involved. Many different jurisdictions have presented guidelines based on THC concentration in biological samples; however. this is a false premise. Indeed, many governments and legal reports have concluded that a conclusive cutoff for THC concentration, which definitively proves impairment, does not exist. See: Sewell, R. Andrew, et al. “The Effect of Cannabis Compared with Alcohol on Driving.” American Journal on Addictions, vol. 18, no. 3, 2009, pp. 185-193., the entire disclosure in which is incorporated herein by reference.

To circumvent the limitations of toxicological screening alone, test operator, including law enforcement, sometimes rely on the examination by a drug recognition expert (DRE). The examination consists of twelve steps intended to seek out signs and biomarkers which are consistent with the presence of impairment. These steps are: (1) breath alcohol test, (2) interview by arresting officer (or employer who suspected impairment), (3) preliminary examination, (4) initial eye examination (horizontal and vertical gaze nystagmus, lack of convergence), (5) divided attention, (6) vital signs (pulse, blood pressure, temperature), (7) pupillometry in a dark room examination, (8) muscle tone, (9) injection sites, (10) subject statements and general observations, (11) opinion of the evaluator, and (12) toxicological confirmation. However, such an extensive examination is a resource-consuming process and takes a significant amount of time before toxicology is performed. In the example of impairment by cannabis products, THC has a very short half-life in serum, and therefore may be eliminated by the time the sample is drawn.

Research tools for studying the pharmacodynamics of D⁹-tetrahydrocannabinol (THC), the main psychoactive compound of cannabis, in combination with the range of cannabinoids, are lacking. The breadth of physiological effects by cannabis products cannot be effectively determined by only examining levels of THC. Other compounds which are present in many strains of C. sativa in pharmacologically active concentrations can modify the activity of THC at its receptors. Further, compounds in cannabis compete with endocannabinoids at receptor binding sites, which could further explain its range of effects in vivo. Cannabinoids can be grouped into three categories depending on their origin: (a) phyto-cannabinoid compounds derived from the Cannabis sativa plant (e.g., D⁹-tetrahydrocannabinol, cannabidiols, cannabinols, etc.), (b) synthetic cannabinoids that are chemically synthetized (e.g., HU-210) and (c) endogenous cannabinoids naturally produced in the body (e.g., 2-AG, AEA). See: Console-Bram, Linda, et al. “Activation of GPR18 by Cannabinoid Compounds: A Tale of Biased Agonism.” British Journal of Pharmacology, vol. 171, no. 16, 25 Aug. 2014, pp. 3908-3917, the entire disclosure in which is incorporated herein by reference. However, the interactions between these various ligands within the cannabinoid class are poorly characterized. These interactions are of interest since they play a role in both acute impairment and the pathology of numerous chronic diseases ranging from drug addiction to cancer. See: Ayakannu, Thangesweran, et al. “Identification of Novel Predictive Biomarkers for Endometrial Malignancies: N-Acylethanolamines.” Frontiers in Oncology, vol. 9, 11 Jun. 2019, the entire disclosure in which is incorporated herein by reference.

Due to the limitations of both current research tools and current testing methodologies used in law enforcement and in the workplace, new approaches to testing must be developed. In the context of impairment testing, such a system would ideally be able to discern ligands that have an active neurological effect on a subject while precluding inactive, structurally similar molecules and metabolites. In doing so it should be able to better characterize the pharmacologic interactions between all cannabinoids (i.e., phyto-, endo-, and synthetic). In addition, it would allow for rapid neurological testing with reliable and reproducible measurements, which could be verified by a third party. Finally, it should analyze multiple inputs and provide the user with a probability of impairment.

These same tools could be repurposed for a number of medical applications including monitoring during drug rehabilitation and overdose detection. Further, such a system could be used to match patients who use cannabinoid medicines with dosing and combinations which maximize therapeutic benefit and/or minimize adverse effects. Such a point-of-collection system would be useful to provide this data in any relevant setting including the point-of-care in a treatment or medical facility and the point-of-sale in a medical dispensary for cannabis.

### SUMMARY

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

Described is a device and method for the collection and interpretation of data for the determination of impairment by intoxicant(s) in a subject at the point of collection. The system relies on the use of multiple sensors which may comprise both a live-cell assay housed in a disposable cartridge for determining the presence of intoxicant(s) and an eye scanning device for determining the vital signs and the neurological state of the subject. The cartridge may be equipped with the capacity to intake a sample, process it, and/or interact it with one or more eukaryotic cell-based biosensors. Further, a cartridge may function as the optical interface for the relay of signals generated by these biosensors to a detector. The eye scanner may be equipped with one or more optical sensors for the detection of both vital signs and neurological state. In the case where multiple optical sensors are utilized, each may be spectrally filtered to identify a biomarker or set of biomarkers. A further method is described which utilizes the eye scanner to train a model to identify additional biomarkers within the live-cell assay on the cartridge to thereby increase the specificity for impairment by the primary analyte. One such example of this approach is to identify combinations of cannabinoids, both endogenous and plant-derived, which when found together with D⁹-tetrahydrocannabinol (THC), confer a higher probability that THC is actively impairing the test subject.

### BRIEF DESCRIPTION OF THE DRAWINGS

By way of example, specific embodiments of the disclosure will now be described, with reference to the accompanying drawings, in which:

FIG. 1A is a flow chart depicting an example of a generalized device workflow for the combinatory assessment of impairment by toxicology (assay device) in addition to neurological and vital signs, where probable cause (defined by neurological impairment) is required to perform toxicology.

FIG. 1B is a flow chart depicting an embodiment of the generalized workflow in FIG. 1A that allows for the simultaneous collection and processing of data by both the neurological state and the toxicology.

FIG. 2 is a flow chart depicting a generalized overview of the model to determine toxicological impairment wherein sensor inputs are initially processed and assigned a score (Tₙ, Vₙ, Oₙ . . . Xₙ) which is further processed and weighed in a final assessment of impairment probability.

FIG. 3A is a flow chart depicting an example of how a score for the toxicology assay may be derived from the sensor inputs in an embodiment where the assay is configured for cannabinoid impairment.

FIG. 3B is a sample output plot for the eye scanner resulting from a pupillary light response test, the output parameters of which can be processed by machine learning as demonstrated in FIG. 2 to determine the presence of impairment.

FIG. 4 is a flow chart depicting a generalized overview view of how the machine learning model is trained to process and weigh the sensor and weighed to determine probability of impairment.

FIG. 5 is a flow chart depicting how the system trains individual machine learning modules by either data generated by the portable tests (toxicology and neurological impairment) or further by data generated from confirmatory testing in a central laboratory, also depicted is a method to train the system to identify intoxicating drug combinations.

FIG. 6A is a perspective view of a single-camera eye scanner with a detachable, sensor-embedded mask.

FIG. 6B is a perspective view of a single camera eye scanner to scan both the left and right eye separately.

FIG. 6C shows the eye scanner's components.

FIG. 6D is a perspective view of the eye scanner as well as describes various sensors and materials used in the mask.

FIG. 6E depicts the form and functionality of the detachable face mask.

FIG. 6F Illustrates the internal components of the eye scanner.

FIG. 6G illustrates additional internal components of the eye scanner.

FIG. 6H depicts a dockable base station for the eye scanner and interface for the test administrator.

FIG. 6I illustrates a top and bottom view of the eye scanner with and without the mask attached.

FIG. 7A is a block diagram showing the components attached to the bus of the eye scanner.

FIG. 7B is a block diagram showing the components attached to the bus of the base station.

FIG. 8 is a flow chart depicting the secure hand-off of network credentials from the base station.

FIG. 9A is a flow chart depicting a generalized overview of the toxicology assay process.

FIG. 9B is a flow chart depicting a more specific workflow of the process of FIG. 9A.

FIG. 9C is a flow chart depicting the same initial steps through sample incubation as in FIG. 9B but allowing for the integration of a wash consisting of ethanol, methanol, or dimethylsulfoxide.

FIG. 9D is a flow chart depicting the integration of cytometry methods into the cartridge in order to improve the sensitivity of the system.

FIG. 10A is a schematic diagram for a cartridge which can perform the sample processing steps illustrated in FIG. 9B.

FIG. 10B is a schematic diagram of a method of adapting a wash into the cartridge layout illustrated in FIG. 10A and for carrying out the handling steps described in FIG. 9C.

FIG. 10C is a schematic diagram showing a method of adapting an additional substrate into the wash in embodiments where the substrate needs to be stored independently of the wash solution.

FIG. 10D is a schematic diagram of an adaptation of the cartridge design to accommodate a cytometry-based approach to improving system performance and sensitivity, carrying out the handling steps described in FIG. 9D.

FIG. 10E is a schematic cross-sectional diagram of a microfluidic chip.

FIG. 11 is a flowchart depicting operation of an optical sensor.

### DETAILED DESCRIPTION

The present embodiments will now be described more fully hereinafter with reference to the accompanying drawings, in which several exemplary embodiments are shown. It will be readily understood that the components of the embodiments as generally described herein and illustrated in the appended Figures could be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in drawings, the drawings are not necessarily drawn to scale unless specifically indicated.

The present inventions may be embodied in other specific forms without departing from its spirit or essential characteristics. The described embodiments are to be considered in all respects only as illustrative and not restrictive. The scope of the inventions is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present inventions should be or are in any single embodiment of the inventions. Rather, language referring to the features and advantages is understood to mean that a specific feature, advantage, or characteristic described in connection with an embodiment is included in at least one embodiment of the present inventions. Thus, discussions of the features and advantages, and similar language, throughout this specification may, but do not necessarily, refer to the same embodiment.

Furthermore, the described features, advantages, and characteristics of the inventions may be combined in any suitable manner in one or more embodiments. One skilled in the relevant art will recognize, in light of the description herein, that the inventions can be practiced without one or more of the specific features or advantages of a particular embodiment. In other instances, additional features and advantages may be recognized in certain embodiments that may not be present in all embodiments of the inventions.

Reference throughout this specification to “one embodiment”, “an embodiment,” or similar language means that a particular feature, structure, or characteristic described in connection with the indicated embodiment is included in at least one embodiment of the present inventions. Thus, the phrases “in one embodiment”, “in an embodiment”, and similar language throughout this specification may, but do not necessarily, all refer to the same embodiment.

In one aspect, the present disclosure relates to detection of pharmacokinetic effects of neurologically impairing substances. One embodiment involves the use of an array of multiple eukaryotic cell-based biosensors expressing receptors on the cell surface for a target set of analytes. More specifically, the embodiment is a method to measure and isolate the active metabolites of the impairing substances and endogenous molecules whose concentrations are directly altered by the presence of the active metabolites of impairing substances. In the case of cannabinoid detection (phyto-, endo-, and synthetic), these receptors include G-protein coupled receptors (GPCRs) including but not limited to CB1, CB2, GPR18, GPR55, and GPR119 (Ye, Cao Et al. 2019). In addition, members of the Transient receptor potential (TRP) ion channels have been identified as cannabinoid receptors. See: Muller, Chanté et al. “Cannabinoid Ligands Targeting TRP Channels.” Frontiers in Molecular Neuroscience, vol. 11, 15 Jan. 2019, the entire disclosure in which is incorporated by reference.

These cell-based biosensors expressing receptors for impairing substances generate an optically detectable read-out linked to a binding event. This can be measured in parallel in a microwell array with benchtop systems in a laboratory (e.g., a fluorometer or luminometer) or in a cartridge system with a portable reader, wherein each well or channel would contain a distinct cell-based biosensor. In one embodiment, the signal is enhanced using flow cytometry, where the cell counts for each receptor following interaction with a sample matrix are measured.

In one embodiment of a multiplexed array, each well or channel consists of a cell sensor for a distinct receptor within a shared class. For example, in a cannabinoid panel one well or channel may contain a biosensor for ligand may would contain a biosensor for ligands of the delta Opioid receptor, another for the kappa Opioid receptor, another for mu Opioid receptor, etc. In another embodiment, parallel wells or channels may contain mutants of the same receptor which have altered binding affinities for a target ligand in a similar manner as has been previously described. See: Ault, Addison D., and James R. Broach. “Creation of GPCR-Based Chemical Sensors by Directed Evolution in Yeast.” Protein Engineering, Design and Selection, vol. 19, no. 1, January 2006, pp. 1-8, the entire disclosure in which is incorporated herein by reference.

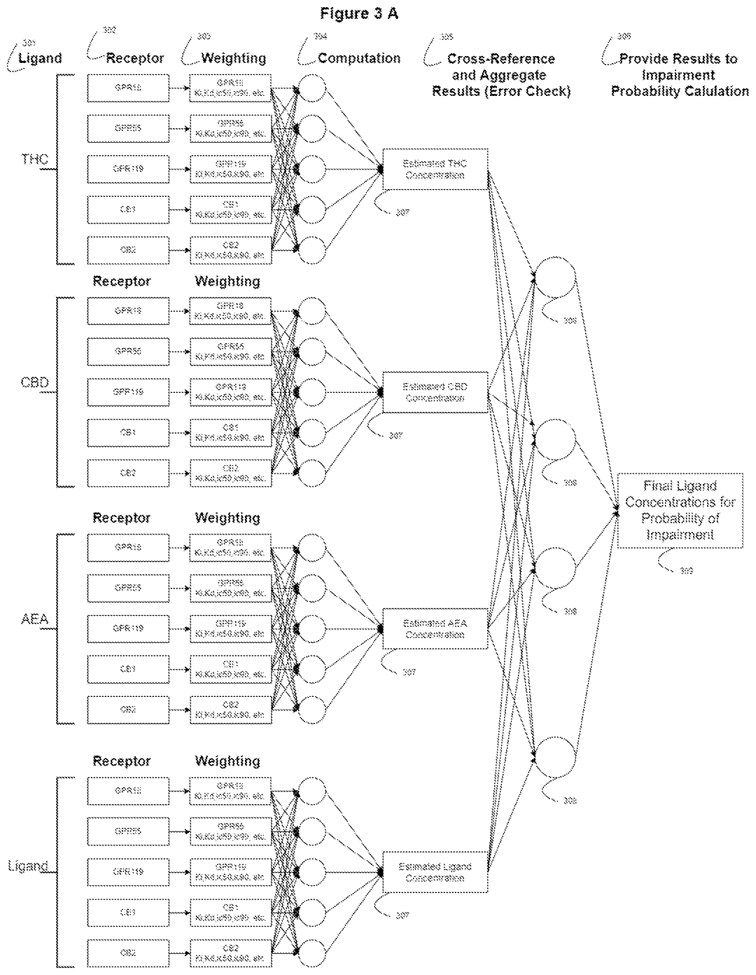

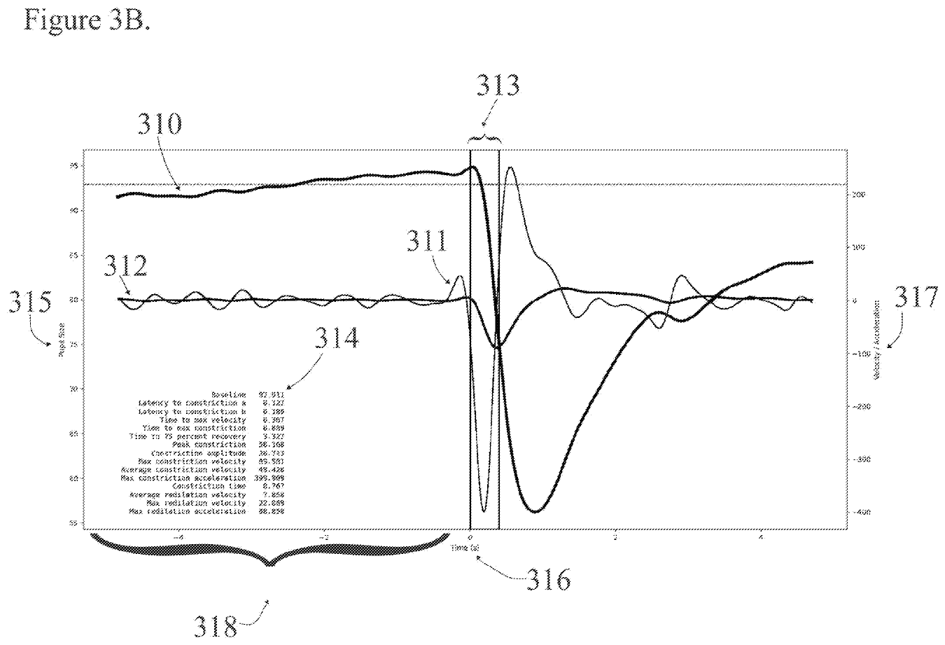

To interpret the results of the assay, the biosensor data from each channel or well is cross-referenced against each other and known parameters of binding dynamics (e.g., Ki, Kd, Ic50, Ic90, etc.). The signal intensity generated by the biosensor may be plotted over time and each of these plots may be compared against one another by a machine learning system. The system may then interpret the plots and produce an estimation of ligand concentrations for the sample, using a weighted system as described in FIG. 3. Training this system may be effected using laboratory reference methods such as GC-MS/MS and compared to the test data generated by the weighted system, wherein the weights may be subsequently altered and optimized with each iteration.

In embodiments where a portable, low-cost reader is required, images of each well may be collected over time and the pixel intensity for each well may be measured. The average intensity across each well may be plotted over time and cross compared. In embodiments where flow cytometry is used, only the cell counts would be cross compared.

One embodiment allows for the measurement of multiple biosensors within the same well or channel. Here, each biosensor variant produces an optical signal of a distinct wavelength, and the results are interpreted by a spectrally filtered imaging system specific to the emission wavelengths. To produce the differentiated optical signals, the receptor binding event may be linked directly to a reporter of a distinct emission wavelength in the biosensor. In embodiments which make use of flow cytometry, the signal may be altered by the presence of a fluorophore inside the droplet housing the individual cell and/or the cell may be incubated directly in the presence of the fluorophore. The well containing the activated droplets may be imaged with a spectrally filtered imaging system.

To improve the accuracy and performance of the assay, the use of controls and reference data stored on chip is described. Here, predicted performance from testing the biosensor batch with an active control may be compared against the results from the control well or channel while running the assay, allowing the system to calibrate its readings. In embodiments which make use of flow cytometry, these controls may be introduced into the droplet containing the biosensing cell. In further embodiments, the control droplets may also be tagged with a fluorophore to distinguish the signal from the active sample.

In another embodiment to eliminate signal noise, the assay may utilize weakly binding ligands with the inverse function of the target analyte in a displacement assay. For example, if the test is designed to screen for an agonist, a weaker antagonist would be selected. In embodiments which make use of flow cytometry, the lower affinity ligand may be introduced into the droplet containing the biosensing cell. A multiplexed assay may make use of any of the described approaches or combinations thereof to improve the performance of the test panel.

Eukaryotic host cells which are capable of functionally expressing a human GPCR at the surface of the cell may be used as the biosensor. These cells may also express trehalose which greatly assists in industrialization steps such as dehydration. Expression of trehalose on the cell wall allows for greater stress tolerance and protection of the sensing proteins which are expressed in the host. See: Iordachescu, Mihaela, and Ryozo Imai. “Trehalose and Abiotic Stress in Biological Systems.” Abiotic Stress in Plants—Mechanisms and Adaptations, 22 Nov. 2011, the entire disclosure in which is incorporated herein by reference. Maintenance of the live-cell assay in a dehydrated active-dry or instant state is a desirable attribute for a commercial product. This allows the cell assay to store in sealed packaging for extended periods of 12 months or greater at room temperature and be utilized on demand off the shelf. For deployment in a laboratory setting such cells may be housed inside a microwell array. For deployment at the point of care, such cells may be stored within a test cartridge containing milli-/microfluidic chip for interaction with a sample matrix.

One such example of a eukaryotic host that may be utilized is Saccharomyces cerevisiae, or more commonly known as baker's yeast. These host cells naturally express two different GPCRs in both the mating and the metabolic pathways. See: Versele, Matthias, et al. “Sex and Sugar in Yeast: Two Distinct GPCR Systems.” EMBO Reports, vol. 2, no. 7, July 2001, pp. 574-579, the entire disclosure in which is incorporated herein by reference. Previous literature has described the expression of human GPCRs to replace one of the endogenous receptors in S. cerevisiae. In addition, these cells have been utilized extensively in industrial processes and are amenable to large batch production. Products based on S. cerevisiae are widely available and sold in an instant or active-dry form. Dried yeast contains high levels of trehalose. See: Plourde-Owobi, L, et al. “Trehalose Reserve in Saccharomyces cerevisiae: Phenomenon of Transport, Accumulation and Role in Cell Viability.” International Journal of Food Microbiology, vol. 55, no. 1-3, April 2000, pp. 33-40, the entire disclosure in which is incorporated herein by reference.

To make use of a dehydrated cell assay at the point of care/collection, a cartridge which functions both as the storage for the cells and the processing for the sample may be used. The described embodiment is intended to be usable by test operators operating in a field environment with minimal to no skill. Therefore, the cartridge must perform the steps normally required of a laboratory technician in a self-contained, automated or semi-automated manner.

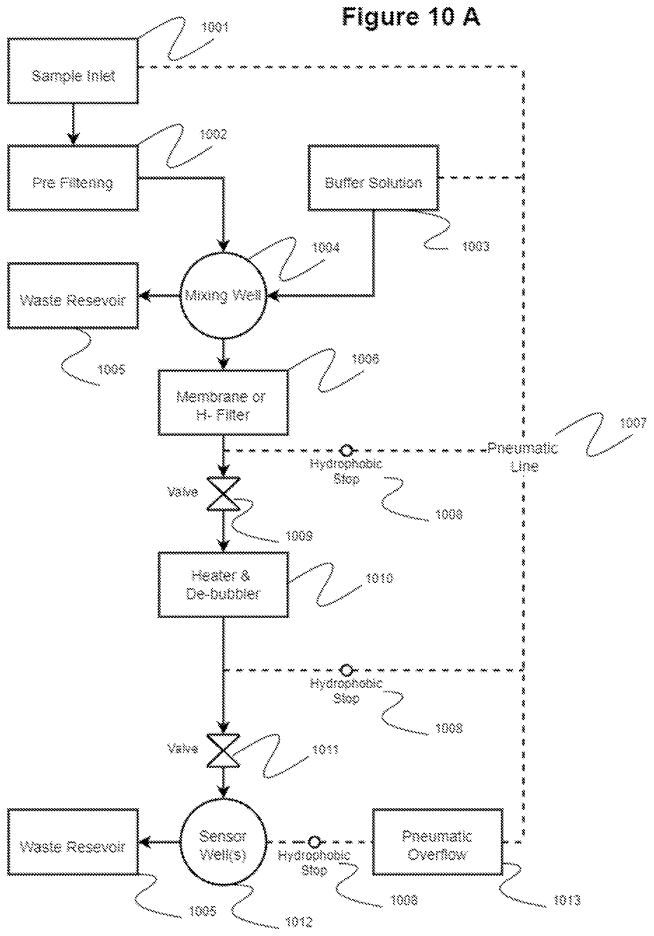

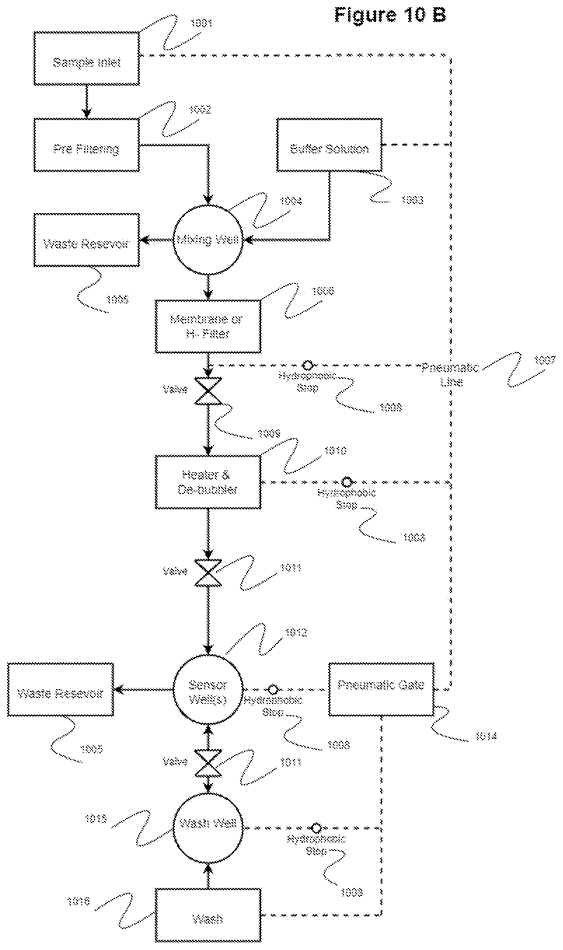

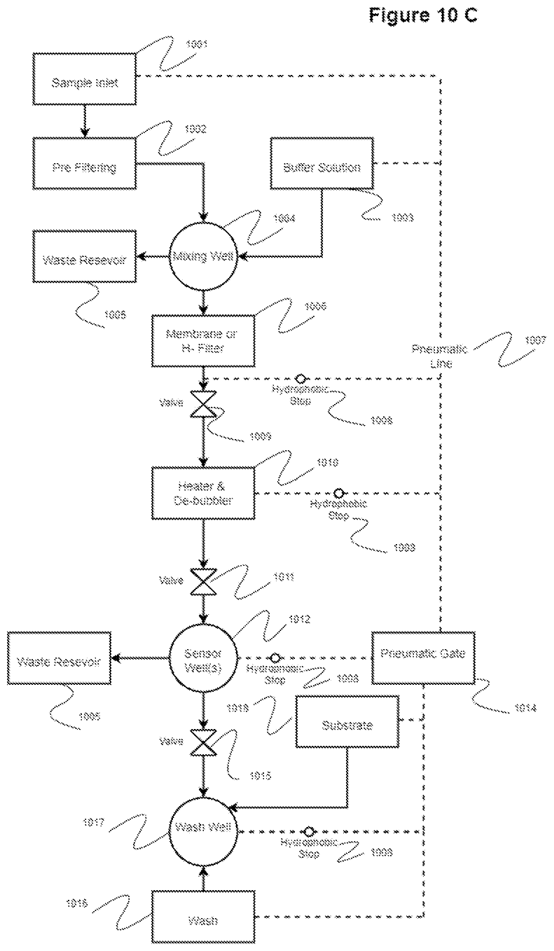

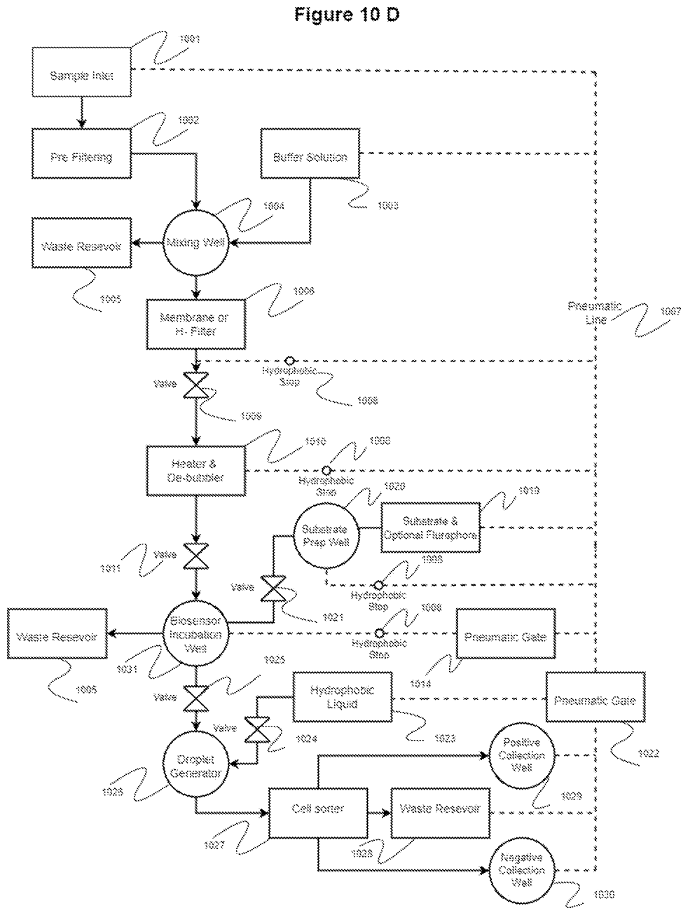

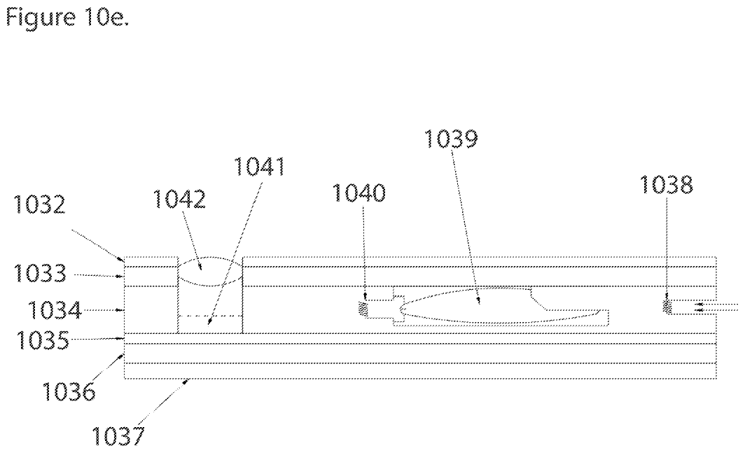

Such a cartridge, as described in FIGS. 10A-10E, would have a unique identifier encoded on the unit itself which is read by the base station. Some examples of unique tags which could be contained on the cartridge include a QR-code, Barcode, Near-field communications (NFC) tag, and/or Radio-frequency identification (RFID). In some embodiments, the information encoded in the tag would provide the base station with information about the contained assay including calibration. The base station assigns the information about the test subject provided by the operator to identifier provided by the cartridge for third party verification. In privacy sensitive applications this information can be encrypted by an unlock key provided to the test operator. In the examples where the tag can support read-write functions instead of just read functions, the test results will be communicated by the base station to the tag for evidence storage.

Following the handshake between cartridge and reader, the collected sample is inserted into the cartridge and secured with a locking or pressure-fit mechanism. After this it interacts with the fluidic system described in FIGS. 10A-10E. The mechanical or pneumatic force provided by the locking or pressing mechanism both releases the sample into the pre-filter and triggers the release of the buffer from a hermetically sealed capsule. The sample is typically mixed with the buffer, warmed and de-bubbled, and finally exposed to the biosensor for analysis. In some embodiments, the pressure provided by this insertion of the sample and/or cartridge provides sufficient pressure for the system to run the complete sample preparation and interaction process. In other embodiments external pumping may be provided automatically by small vacuum pumps or manually with finger pressure by the test administrator.

The sensor well itself may contain: (1) the dehydrated cell sensor, (2) a rapidly dissolvable adhesive bound to the dehydrated cell sensor (or other immobilization approach), (3) a metabolic sensor to measure activity of the cells upon rehydration, (4) an optical interface for accepting excitation (as needed for fluorescence) and transmitting sensor response with the described optical detector, and (5) an optional means of mixing or homogenizing. In some embodiments where multiple wells are desired the filtered sample is evenly divided into these wells by the fluidic array.

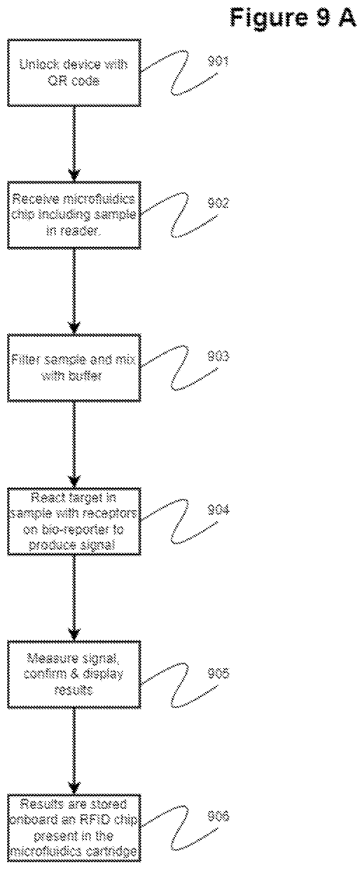

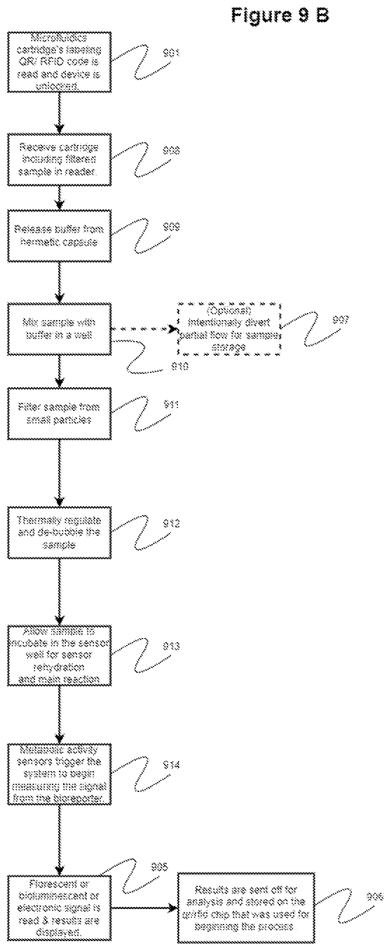

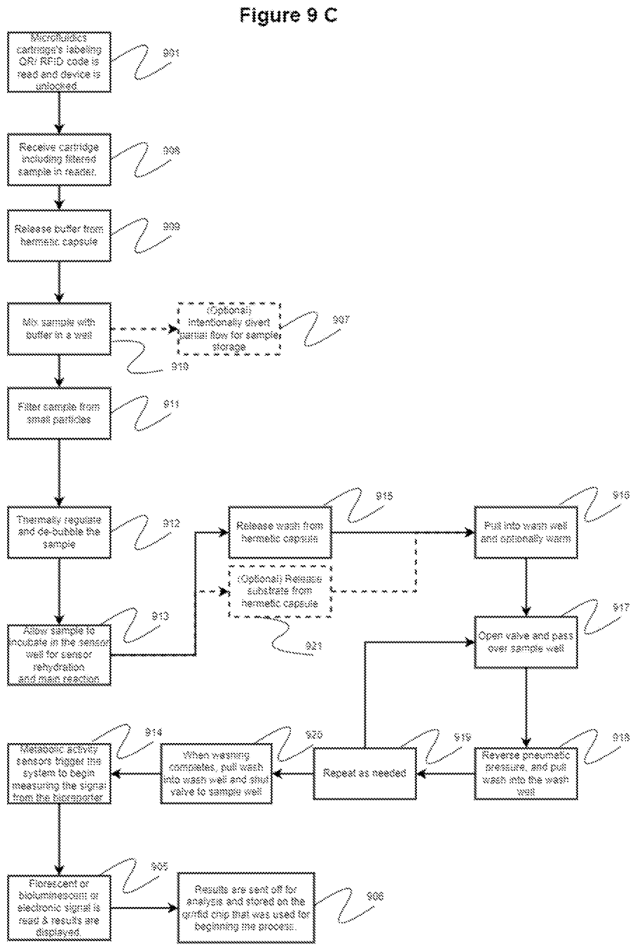

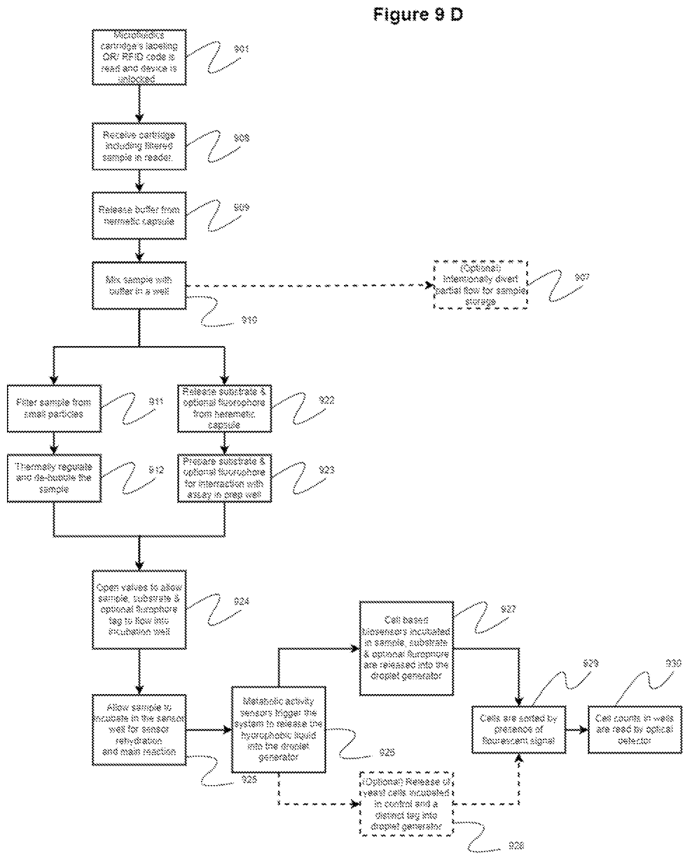

In any of these embodiments, it may be desirable to include a wash solution (FIGS. 9C, 10C) in a hermetically sealed capsule which can be passed over the well multiple times before interaction with the sample. In some embodiments the force may be applied externally (e.g., by the test operator) or with a pump system, in either case, the force would be controlled by the pneumatic gate with alternating pressure applied to the well containing the wash and the sensor well. Examples of wash solutions include ethanol, methanol, and dimethylsulfoxide which can suspend the substrate with minimal impact on the viability of the cells.

The chosen adhesive must have a low moisture content to preserve the dry state of the cells. In addition, it needs to be biocompatible and quickly dissolvable upon rehydration with the sample and/or wash. In one embodiment where saliva is used, this adhesive can be starch based which makes use of the amylase naturally present in the test sample. Amylases enzymatically process glucose-based polymers such as starch into smaller maltose molecules enabling the adhesive to be utilized as metabolic fuel for the yeast upon hydration.

Each well will contain a metabolic sensor which will trigger once the cell sensors in the well are ready to produce an optical signal following rehydration. This can include a system to detect a single or combination of metabolic products of the host cell. To ensure reproducible rehydration times and even distribution of analytes, sensors, and/or substrates in each sensing well a mixing approach can be applied to each well. This can include the same approaches for the initial mixing well: sonication, centrifugation, magnetic bead assisted, or passive (based on flow).

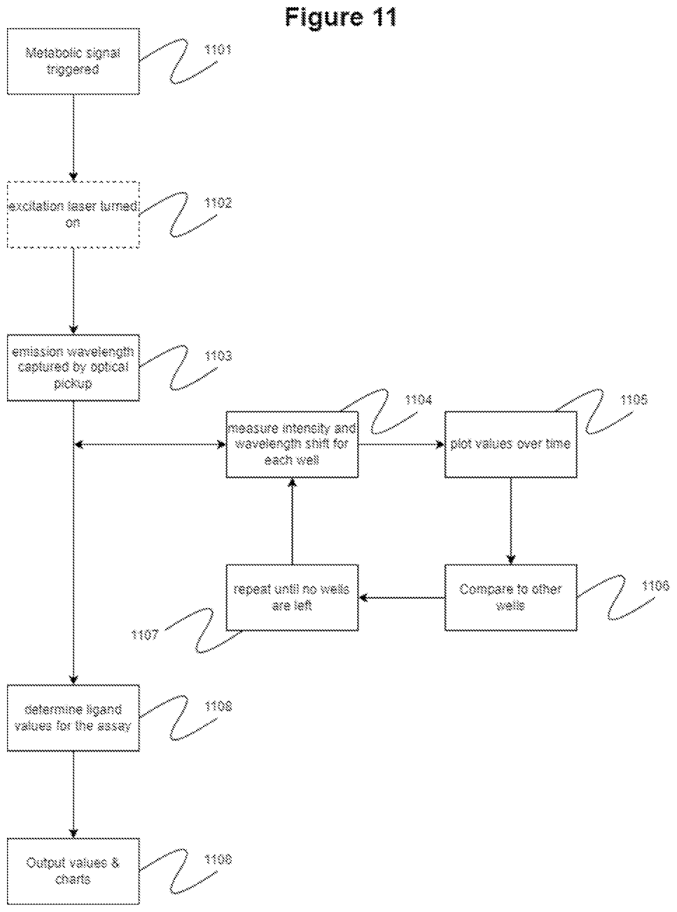

Once the metabolic sensor is triggered, signaling the biosensing cells are ready for analysis, the system will turn on the optical detector FIG. 11. In embodiments where the biosensing cells utilize a fluorescent reporter, this will also provide power to the excitation laser optimized to the properties of the reporter. Further the fluorescent embodiments of the invention may utilize a color filter which isolates the emission wavelength from the excitation wavelength allowing for greater signal fidelity.

In embodiments where the biosensing cells utilize a luminescent reporter, no such excitation laser is required. Instead, the system will rely on the presence of a substrate to produce a luminescent signal. Examples of substrates for a luminescent reporter system include furimazine, luciferin, and coelenterazine. These can be further modified for additional solubility and stability such as has been described for furimazine: hydrofurimazine, fluorofurimazine, and O-acetylated analogs. See: Gaspar, Natasa, et al. “Evaluation of NanoLuc Substrates for Bioluminescence Imaging of Transferred Cells in Mice.” Journal of Photochemistry and Photobiology B: Biology, vol. 216, March 2021, p. 112128, the entire disclosure in which is incorporated herein by reference.

In some embodiments the substrate is stored directly inside the host cell prior to dehydration. In some embodiments the optical biosensing mechanism requires the use of an external substrate to enhance the signal. For embodiments which make use of an external substrate, a variant of the design is described in FIGS. 9C, 10C Here, an additional hermetically sealed capsule contains the substrate suspended in a liquid which is released by the pneumatic pressure in the system. In another embodiment, a lyophilized substrate can be included inside the sensor well with the dehydrated cell sensor. In another embodiment, the provided pneumatic pressure can come from thumb force applied to a squeeze capsule.

The signal generated by the cells will be captured by an optical detector over a period of time that is experimentally determined to be optimal for the biosensors. Each well is capped with an optically translucent material, which in some embodiments is provided with a polarized and/or color filter. Further, well(s) may have a reflective backing, such as through a biocompatible foil. This allows for the capture of more of the optical signal generated by the biosensor. In further embodiments the window may be positioned directly in line with a micro-lens array housed in the reader, these lenses may be fused to an optical fiber for the isolation of each well's signal in a multiplexed array. In some embodiments, a photomultiplier may be used to enhance the biosensor signal.

A cross-sectional schematic (not to scale) is provided in FIG. 11. The cartridge may consist of a cellulose-based layer that is adhered to a reflective metallic foil, which forms the seal for an open-backed fluidic chip. An adhesive is applied in a pattern so as to match the fluidic channels in the plastic layer. This plastic layer can be formed out of polydimethylsiloxane (PDMS), cyclo-olefin co-polymer (CoC), polycarbonate (PC), UV-curable resin, or any other suitable material for milli-/microfluidic fabrication. An additional pneumatics layer is made from similar materials as described for the fluidic layer, interfaces between the pneumatics and fluidics are separated by a hydrophobic barrier to prevent cross contamination and chip failure. Finally, a cover is bonded to the top of the chip to form a seal and ensure function of the channels, this cover may contain a cut-out or optical window for the assay wells to interface with an optical pick up (not shown). The well may in some embodiments be capped with a microlens to focus the signal to the pickup and allow for multiplexing.

Fabrication of the fluidic and pneumatic plastic layers may be performed by some combination of 3-D printing, milling/machining, and/or soft lithography. In some embodiments the fluidic chip may be directly fabricated with 3-D printing such as through stereolithography or fused deposition modelling, the printed chip would be open-backed and amenable to surface coating (e.g., spin, vapor, and/or sputter) to remove impurities and biofouling. In one embodiment a biocompatible photoresist is used such as SU-8. While in other embodiments a negative mold can be fabricated through the same 3-D printing processes for casting with a suitable polymer such as PDMS. In some embodiments the microfluidic components are produced in a typical soft lithography method while the milli-fluidic components are milled.

### Eye Scanning Headset:

In addition to the assay, an eye scanning headset is described to perform an assessment of a subject's vital signs and neurological state. These data provide context to the toxicology results obtained from the assay and in combination lead to a weighted probability of impairment (as a positive case) or fatigue (as a negative case). The initial eye scanning tests may be performed simultaneously with the processing of the toxicology assay.

These initial assessments may consist of the standard validated measures employed by officers and DREs today. These can broadly be broken down into two categories—vital signs and ocular measurements. Vital signs may include: (1) heart rate, (2) respiratory rate, (3) blood pressure, and/or (4) temperature. Ocular measurements may include: (1) lack of convergence, (2) pupillary hippus, (3) rebound dilation, (4) vertical gaze nystagmus, and/or (5) horizontal gaze nystagmus.

To obtain data on a subject's vital signs, this invention makes use of photoplethysmography (PPG). One embodiment of this invention relies on PPG measurements from multiple sites on the subject's face simultaneously. Multi-site PPG has been previously described to measure additional features of the pulse, beyond just the heart rate, including pulse transit time, a measure highly correlated with systolic blood pressure. See: Chan, Gabriel, et al. “Multi-Site Photoplethysmography Technology for Blood Pressure Assessment: Challenges and Recommendations.” Journal of Clinical Medicine, vol. 8, no. 11, 1 Sep. 2019, p. 1827; and Elgendi, Mohamed, et al. “The Use of Photoplethysmography for Assessing Hypertension.” Npj Digital Medicine, vol. 2, no. 1, 26 Jun. 2019, the entire diclosures in which are incorporated herein by reference. In some embodiments PPG measurements may be taken by contact probes on the branches of the carotid artery, on the supratrochlear vessels, and/or branches of the facial artery such as the angular artery. In embodiments which make use of auditory stimulation, some of these probes can be placed on the earpiece of the headset.

In another set of embodiments, PPG is measured in the same arteries through mapping oxygenated and deoxygenated hemoglobin with spectrally filtered cameras or a hyperspectral camera operating within 500-660 nm range. This contact-less approach can additionally be applied to imaging ocular blood vessels, including those found in the retina, choroid, and sclera. The camera module(s) would collect a time series of snapshots, which is preferrable in this application to line-scanning.

In addition to PPG measurements, the hemoglobin maps can be analyzed for the determination of irregular vasodilation/vasoconstriction patterns in the imaged vessels which have a direct impact on local thermal regulation. Literature has previously described temperature irregularities in the face and eye as characteristic of impairment by alcohol, See: Kubicek, Jan, et al. “Prediction Model of Alcohol Intoxication from Facial Temperature Dynamics Based on K-Means Clustering Driven by Evolutionary Computing.” Symmetry, vol. 11, no. 8, 3 Aug. 2019, p. 995., and Koukiou, Georgia, and Vassilis Anastassopoulos. “Drunk Person Screening Using Eye Thermal Signatures.” Journal of Forensic Sciences, vol. 61, no. 1, 22 Dec. 2015, pp. 259-264, the entire disclosures in which are incorporated herein by reference. Further cannabis has known effects on thermal regulation as well as blood vessel dilation. Therefore, analysis of these parameters in facial and/or ocular imaging may be beneficial in determining the presence of impairment.

In some embodiments, cameras operating within the infra-red spectra may be utilized to determine the facial or ocular temperature map. Data from these infra-red cameras can be analyzed together with the hemoglobin maps to add greater confidence.

To obtain pupillometry data, a standard hardware configuration consisting of a near-infrared light source and image detector(s) operating across the visible light and near-infrared spectra are utilized. In one embodiment, this hardware is housed in a binocular form factor to isolate the eyes from ambient light stimulation and simulate a dark room, typical of DRE exams. In one embodiment, multi-spectral or hyperspectral imaging in the infrared spectra of the Iris sphincter and dilator muscles is used to provide greater detail and resolution of the sympathetic/parasympathetic pupillary response. This analysis can extend to the pupillary dark response (sympathetic nervous system) beyond just the standard light response (parasympathetic nervous system). In one embodiment, only one camera is needed to obtain measurements from both the left and right eye.

A visible light stimulus in a sequence of varying intensities may be utilized in order to perform the pupillary light response exams including rebound dilation and hippus. Parameters of pupillary response measured include response latency, maximum constriction, pupil escape and pupil recovery phases. In one embodiment the stimulus is provided for by an image display which can simulate an object moving in order to perform the lack of convergence and horizontal/vertical gaze nystagmus exams.

During these stimuli, a timestamped series of snapshots is captured by the image detector correlated with the vital sign measurements and analyzed in aggregate. A machine optimized algorithm for the detection of the pupil radius in each snapshot is utilized to determine changes in response to stimuli. Further processing can be applied to detect blink frequency during examination and eliminate data irregularities. In embodiments of the test sequence which rely on positional tracking, relative locations of the pupillary circumference will be measured between snapshots to determine position and seek out irregularities consistent with standard test parameters.

In more specific tests for impairment, use of psychopharmacologic tests may be employed including attention tasks, awareness tasks, and error detection. The stimuli for these tests be provided in visual and/or auditory form. One embodiment makes use of matching the frequency of pupillary oscillations with the frequency of the visual stimuli to determine attention tracking as described previously. See: Naber, Marnix, et al. “Tracking the Allocation of Attention Using Human Pupillary Oscillations.” Frontiers in Psychology, vol. 4, 10 Dec. 2013. Another embodiment makes use of sustained attention tasks to determine awareness or mind wandering, again within the context of pupillary response as described in literature (Unsworth, Robison 2018). A further embodiment makes use of pupillometry in response to audio stimuli to measure the dilation response to speech as has been described. See: Wang, Yang, et al. “The Pupil Dilation Response during Speech Perception in Dark and Light: The Involvement of the Parasympathetic Nervous System in Listening Effort.” Trends in Hearing, vol. 22, 2018, p. 233121651881660. In the context of any test, a time series of vital signs as described above will be matched with the test sequences and pupillary response to gain a more complete picture of the autonomic responses during these stimuli.

Another embodiment makes use of active feedback from the test subject during the stimuli. Parameters including response accuracy and latency can be used to provide greater detail on the neurological state of the subject including the somatic nervous system response. The timestamped data collected by the voluntary and involuntary measures of neurological response may be analyzed in aggregate to improve the accuracy of the system to determine neurological impairment.

As noted above, the subject matter described herein includes methods and systems for sample collection, evaluation, and analysis of the presence of neurological impairment by one or more intoxicating substances. The overall system may make use of a combination of one or more of the devices for collection of biomarkers described and/or commercially available devices which collect data from similar biomarkers. For the scope of this disclosure, biomarkers may be defined as pharmacokinetic and pharmacodynamic data points of an intoxicating drug's activity. Pharmacokinetic data points may consist of quantifiable levels of intoxicant metabolites and/or endogenous molecules which are directly altered by the presence of intoxicant metabolites found in a sample matrix. In a preferred embodiment, the sample matrix is derived from saliva, but other embodiments may also derive from other matrices used in toxicology including, but not limited to: blood, sweat, urine, breath and hair. Pharmacodynamic data points may consist of signatures of neurological function including, tonic and phasic pupil size, eye movement, vital signs, and motor coordination. This pharmacodynamic data may be collected in the context of variable light stimulation as is the case in a typical pupillary light response test, or during specific tasks and test sequences described herein.

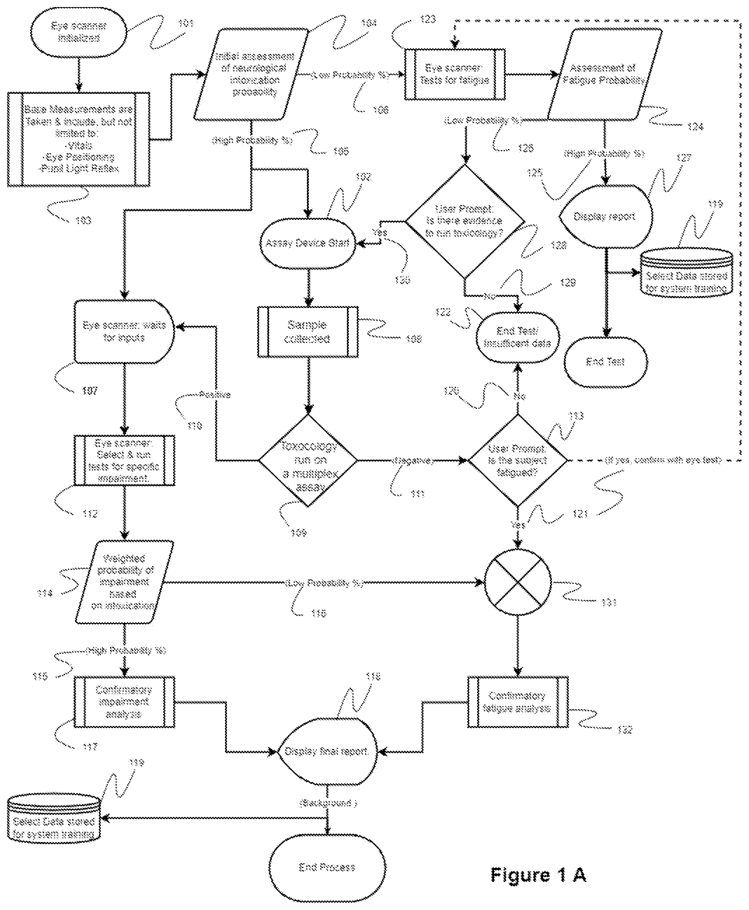

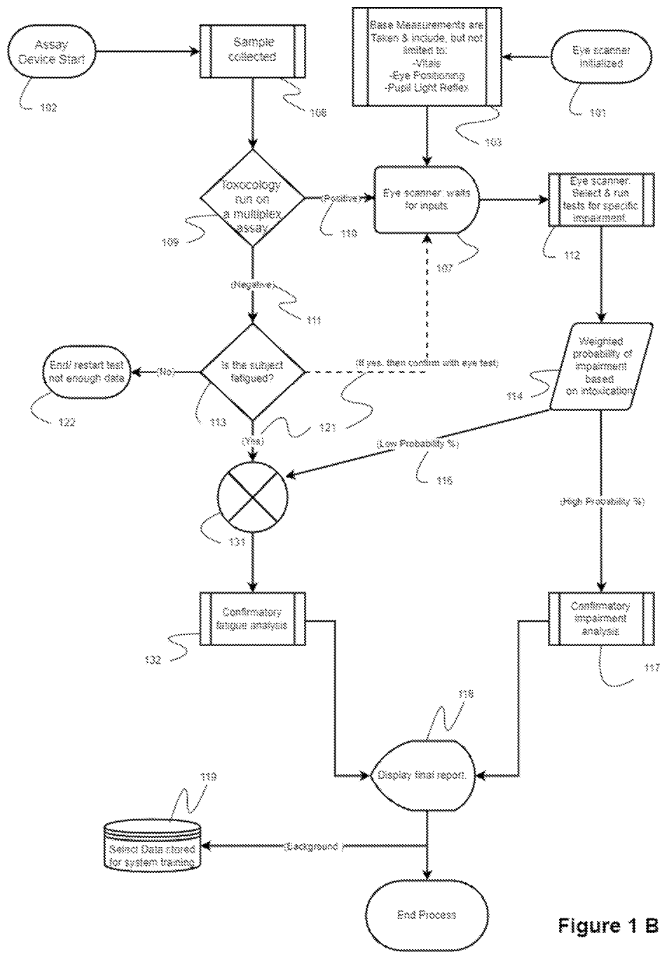

A generalized method for the collection and analysis of the pharmacokinetic and pharmacodynamic data points by two devices, an eye scanner for pharmacodynamics 101 and a toxicology assay for pharmacokinetics 102 is described in FIG. 1A and FIG. 1B. FIG. 1A more specifically describes the sequential testing procedure to collect pharmacodynamic data before pharmacokinetic data. This may be desirable in jurisdictions requiring pharmacodynamic measures of probable cause to perform toxicology. FIG. 1B describes a testing procedure where pharmacokinetic and pharmacodynamic measures are collected and interpreted in parallel, to reduce the time required to complete the impairment analysis.

In the procedure described in FIG. 1A, the pharmacodynamic tests are conducted by the eye scanner 102 in this example to perform an initial assessment of impairment probability 104, through the base measurements collected during the initial assessment 103. This initial assessment 103 will evaluate pharmacodynamic datapoints which are frequently assessed during the police drug recognition expert (DRE) examination procedure. This includes vital signs such as heart rate, blood pressure, and respiratory rate as well as the pupillary light response and pupillary positioning tests. For the pupillary positioning tests, this may consist of Lack of Smooth Pursuit (LOSP), Lack of Convergence (LOC), and 45-degree Horizontal Gaze Nystagmus (HGN). In embodiments where the detection of cannabis impairment is required, the pupillary light response will be analyzed for the presence rebound dilation in addition to hippus.

If the probability of impairment is determined to be low 106, then the eye scanner begins test and task sequences specific for the detection of fatigue 123. These tests and tasks may consist of oculomotor tests including optokinetic nystagmus (OKN) and attention-driven pupil response. Further tasks requiring more direct feedback by the test subject may in some embodiments be performed and include error detection, reaction time, critical tracking, divided attention, and psychomotor vigilance. In a preferred embodiment, the selection of these tasks may be performed by a deep learning system.

An assessment of fatigue probability 124 is made, leading to a fatigue analysis report 127 if the probability was deemed to be high 125. If the probability was deemed to be low 126, then the test administrator is prompted to begin toxicology 128. The test administrator can opt out of performing toxicology 129, if for example there is a lack of sufficient evidence to do so. Otherwise, the user prompts 130 the toxicology sequence 102 and proceeds to collect the sample 108.

If the probability of impairment is determined to be high 105, then the eye scanner or other pharmacodynamic is placed on standby 107 while the assay 102 test sequence is initiated. A sample matrix from the test subject is collected 108 by the test administrator and processed by the toxicology assay 109. In embodiments which make use of the point of collection cartridge system described in FIG. 10A-D, the procedure for this is outlined in FIG. 9A-D and will be detailed further in the disclosure.

Based upon the findings of the toxicology assay, the eye scanner will either run specific tests for impairment 112 for positive findings in the toxicology assay 110 or prompt the user 113 in the case of a negative finding 111. Following the specific impairment tests 112, a weighted probability is assigned 114.

If the user inputs that the subject is not fatigued 120 during the prompt 113, then the test is ended, and the device interface displays an error for insufficient data 122. If the user inputs that they have reason to believe the subject is fatigued 121, this is confirmed with a test and task sequence specific for the finding of fatigue 123. The specific tests and tasks performed for impairment 112 are based upon similar tests and tasks performed for the detection of fatigue 123 but analyzed by the system in a way that is optimized for impairment detection.

If the probability of intoxication assigned by the pharmacodynamic test analysis 114 is determined to be high 115, then a confirmatory impairment analysis 117 is performed which aggregates all pharmacokinetic and pharmacodynamic data gathered and analyzes for specific drugs of impairment. If the probability assigned by the pharmacodynamic test analysis 114 is determined to be low 116, then the data from the tests performed and user input 121, if applicable, are aggregated 131 and analyzed for the specific presence of fatigue 132. In either result of the test analysis 114, a final report 118 is displayed, and select data gathered is stored 119 for training one or more of the computation layers and processors described herein.

In FIG. 1B, a procedure is described to simultaneously collect pharmacodynamic data and pharmacokinetic data from devices and sensors. In one embodiment, the toxicology sample 108 is first collected and immediately after being placed into the assay 109 for analysis, the test administrator would begin 101 to collect the initial pharmacodynamic parameters. For this disclosure the methods described for each module 103 and 109 are consistent across FIGS. 1A and 1B.

If the findings of toxicology are positive 110, then the eye scanner will select and run test sequences 112 specific for the form of impairment caused by the intoxicants determined by the assay. The selection of tests 112 will be further informed by the results of 103. If the findings of toxicology are negative 111, then the test administrator is prompted for fatigue 113. If the test administrator does not believe the test subject is fatigued, the system displays an error for not enough data 122. If the test administrator believes the test subject is fatigued, this input is weighed in the test selection 112 along with the results of the initial measurements 103.

In either case, the weighted probability of impairment as a result of intoxication 114 is established and as a result, is either aggregated with test administrator feedback 131 and analyzed for fatigue if intoxication is deemed unlikely or confirmatory analysis for the specific drug(s) of impairment 117 is performed. Both will produce a final report 118 and trigger the system to store data for further training of training one or more of the computation layers and processors described herein.

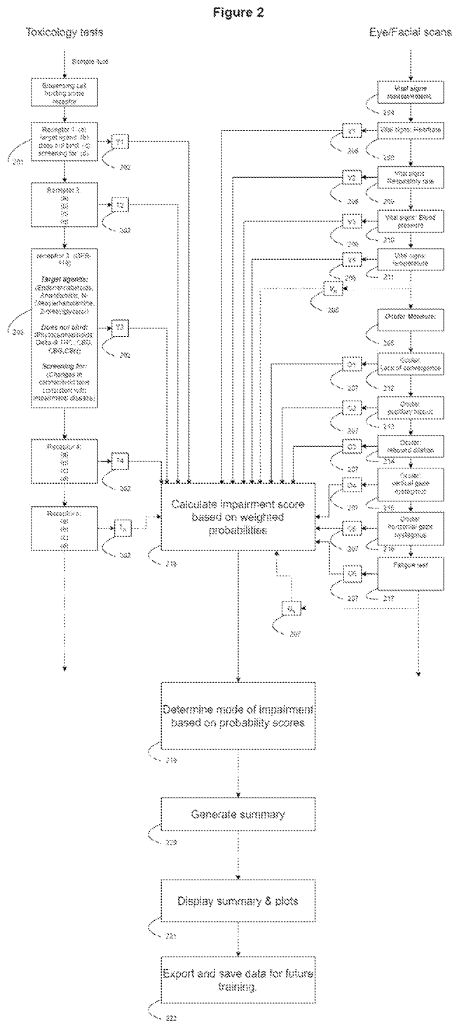

In FIG. 2, a system is described which aggregates all the pharmacokinetic and pharmacodynamic inputs from sensors. Here, the described machine learning instance(s) function(s) as a computational layer on the data collection device(s) housing sensors (The sensor stack) for biomarkers and provides an estimate of the probability of impairment. In this disclosure, biomarker measures are processed through machine learning functions Tₙ 202 for pharmacokinetics and Vₙ 206 and Oₙ 207 for pharmacodynamics (vitals and ocular measures, respectively). The machine learning instance(s) may consist of multiple sub processes.

In one embodiment described in FIG. 2, the pharmacokinetic analysis is measured through interpretation of multiple receptor binding assays. Here the properties of each of the receptors are known and provided as inputs for a machine learning function, Tn 202. These factors include the target ligands for that receptor, their function at that receptor (e.g., agonist, antagonist, partial agonist, inverse agonist, etc.), and excluded ligands for the receptor 201. A qualitative example is provided for GPR119 203, which in this instance is a receptor well suited to screening endocannabinoid activity while filtering out phytocannabinoids. The machine learning instance will also gather data from the receptor binding assays 109 and may consist of further sub-processes for the analysis of this imaging data.

These sub processes under Tₙ 202 may make use of machine vision tasks to improve the signal fidelity of the data generated by the collected matrix interacting with one or more biosensors. In a preferred embodiment where the sample matrix interacts with a live cell assay in a well or channel, images of the cells are collected in a series of snapshots over time. Classifiers may be assigned to these snapshots. In embodiments where more than one well is captured by an imaging device, individual tiles may be assigned to each well's signal and classified accordingly. For each of these snapshots, each pixel is analyzed for brightness intensity, and if applicable, spectral data. The machine vision process would interpret this aggregate data and produce an initial estimate of ligand concentration. In embodiments which make use with multiple live cell assay interactions in parallel, referred to as a multiplexed assay, another computational layer may be applied which cross references the aggregated data from each assay and adjusts the estimated ligand concentrations accordingly as described in greater detail in FIG. 3A. In some embodiments the multiplexed assay contains control channels which functions as a reference input for the overall interpretation of data.

In an implementation, a deep learning framework is utilized to periodically optimize the machine learning instance present on the device(s) for the analysis of pharmacokinetic data, Tₙ 202. This deep learning framework collects data from multiple sources including, but not limited to: the aggregated data from deployed devices for collection of biomarkers, reference data points generated through confirmatory testing, supervised feedback, and published data sources. In one embodiment, the deep learning framework optimizes the computational layers for the pharmacokinetic datapoints by comparing the test results against confirmatory testing in a laboratory through accepted measurement protocols, for example gas chromatography and mass spectrometry, functioning as the training data in the model. In a further embodiment, the analysis of well imaging data can be performed by comparing the binding activity against known receptor binding kinetics and the correlation coefficient is calculated through typical nonlinear methods (e.g., Spearman's rho).

In a further embodiment, the values of ligand concentrations themselves will be assigned a coefficient or weight in the other all interpretation of test results. The assigned weighting will be a function of aggregated prior test results which in some embodiments be analyzed by a deep learning system trained to isolate the signatures of impairment. Toxicological findings strongly correlated or inversely correlated with impairment will be assigned a greater absolute weight for example than findings which are weakly correlated. This weighted score approach can be combined with a rules-based approach which accounts for the other inputs to the system from the pharmacodynamic measures. The machine learning analysis may also be performed in a typical binary tree structure. The presence of an initial condition, for example activation of a certain receptor, must exist, in order for the defined next set of conditions to be assessed by the system. Other parameters can be applied to the binary tree based upon pre-specified quantifiable or qualifiable thresholds. In some embodiments these thresholds are adjusted by the deep learning analysis.

Further inputs for the system described in FIG. 2 include vital signs 204 and ocular measures 205, with each assigned weights for their respective test inputs Vₙ 206 and Oₙ 207. The vital signs may include heart rate 208, respiratory rate 209, blood pressure 210, and temperature 211 and can be gathered by any combination of PPG, electrocardiography, spectral imaging, or other accepted method to do so. In an embodiment, the vital signs may also extend to include other direct measurements of neurological activity, for example electroencephalography (EEG) and functional Near-Infrared Spectoscropy (fNIRS). In some embodiments a time variable is introduced into the analysis and these measures are taken relative to the test or task sequence employed by one or more devices. The tests described for the ocular measures 205 include but are not limited to: Lack of Convergence 212, pupillary hippus 213, rebound dilation 214, vertical gaze nystagmus 215, horizontal gaze nystagmus 216, and a test for fatigue 217. To perform these analyses, particularly on the measures which are dependent pupillary size and light reflex (e.g., pupillary hippus 213 and rebound dilation 214), FIG. 3B demonstrates an example of test results from an eye scanning device. In some embodiments, the measurements and parameters derived from these test results are directly used as inputs for the ocular measurements 205 in the impairment score calculation 218.

The probability of intoxication 114 and fatigue 124 based upon the pharmacodynamic parameters 204, 205 can in some embodiments be optimized by a combination of one or more computation layers and processes 206, 207. These sub processes may make use of machine vision tasks including: identification of pupillary location and outline, size measurements, and feature extraction from a series of snapshots collected from the eye and surrounding facial regions. In an embodiment, these snapshots are tagged with data collected from vital sign sensors. In an embodiment, the deep learning framework assigns the coefficient or a weighted score Tₙ 202, Vₙ 206, and Oₙ 207 to the datapoints collected by the data collection device(s) in order to optimize the impairment or fatigue probability recognition by the machine learning system. In a further embodiment the deep learning framework optimizes the parameters of the test and task sequences 112, 122 based upon the weighting assigned, feedback regarding fatigue, and the status of identified intoxicants during toxicology screening.

FIG. 3A presents an example analysis Tₙ 202 method for determining the levels of ligand concentrations from multiple receptor binding assays. Here the system is given a series of ligand targets 301 to look for. The system is informed of which receptors are present 302 and has a known set of binding kinetics for those receptors to assign a weight for analysis 303. An initial computation layer(s) is applied specific for the analysis of that ligand's concentration 304 and is optimized by one or more machine learning and/or deep learning processes. In some embodiments the optimization of these computation layer(s) is trained by reference confirmatory lab data derived from systems such as GC-MS/MS. A second analytical layer 305 is described where the findings of each ligand's concentration 307 are cross-referenced against one another and another computation layer or layers is applied to this analysis 308 to achieve the final ligand concentrations 309.

FIG. 3B is an example output of the eye scanner system following a pupillary light response test, which displays the pupil size 310, velocity 311, and acceleration 312 in a plot over time. The left y-axis represents the pupillary size 315, the x-axis represents time 316, and the right y-axis represents velocity and acceleration 317. The system first records baseline measurements in a simulated dark environment 318, then is exposed to visible light in a specific window starting at time 0 on the x-axis 313, and the resultant response is analyzed. The analysis is displayed in the table 314, whereby various parameters including latency, time to max velocity, time to max constriction, time to 75 percent recovery, peak constriction, etc. are determined. These parameters can all function as inputs 205 and assigned a weight Oₙ 207 for the system described or can be used to derive the measurement inputs such as hippus 213 and rebound dilation 214 with computation layer(s) applied.

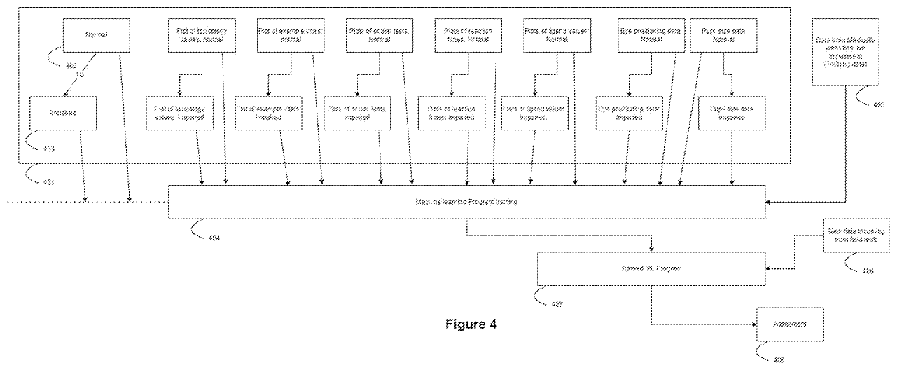

FIG. 4 is a generalized example of how the weights and computation layers described in FIG. 2 are determined and an assessment is made. Aggregated data 401 from all sources (Toxicology, Ocular, and Vital) has been assigned a label or classification of either normal 402 or impaired 403 is fed into the machine learning training 404. In addition, data from supervised classification, medical sources (e.g., clinical studies), published data, confirmatory lab analysis 405 can be fed into the machine learning training 404 to optimize one or more computation layers. When combined with uploaded data from field deployed tests 406, the system is continuously trained and optimized for analysis of that data and classification into impaired or normal. In some embodiments the method described herein can be applied to the classification of fatigue. The identification of fatigue is then treated as an input for the machine learning classifier of impairment to further improve the system. In some embodiments the medically classified data 405 also includes data from those with neurological disease to be treated as an exclusion classification from the determination of impairment.

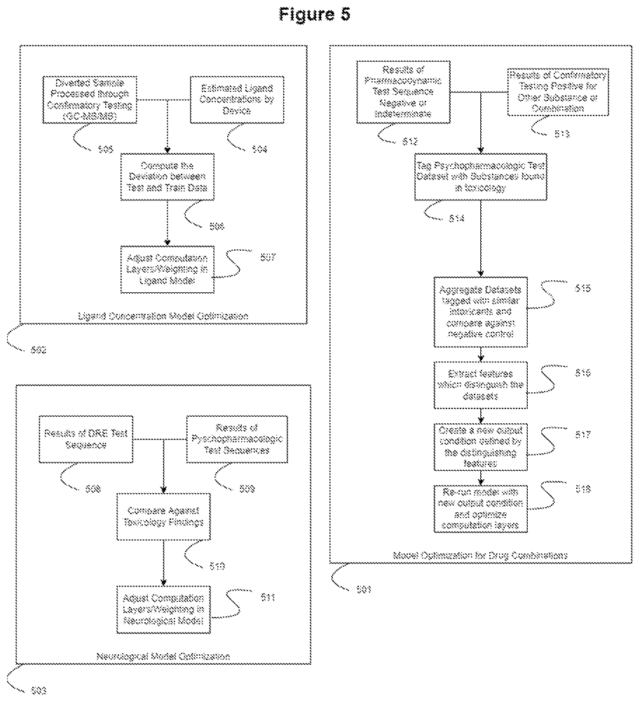

FIG. 5 is a generalized example of how different computation layers may be trained by additional reference data or system data. The ligand concentration model optimization 502 utilizes the estimated concentrations by the field deployed system 504 as the test data and the confirmatory lab analysis of the diverted 505 as the train data in a typical machine learning workflow. The differences between the two analyses are determined 506 and the machine learning algorithm is then altered by changing the parameters of its computational layers including weighting and rules to reduce the differences between the test and training data inputs. These new parameters are tested against the existing body of test and training data to ensure that the differences remain within an acceptable margin of error.

The neurological model is optimized 503 by taking into account the pre-defined and fixed test parameters in the drug recognition expert (DRE) test protocol for pupillometry 508 as well as the results of the dynamic psychopharmacologic tests with variable test parameters 509. Both of these inputs are weighed against the toxicology findings 510 in order to determine the weights that the fixed 508 and dynamic 509 tests should be assigned to improve the system's sensitivity and specificity for specific impairment types. For example, the DRE protocol has been shown to have sub 80% sensitivity for cannabis impairment when performed by trained agents, the weights assigned to the dynamic tests 509 would be assigned to improve this sensitivity to above 90%. In some embodiments the parameters of the dynamic tests are adjusted by one or more machine learning layers in order to improve the system performance.

The model optimization for drug combinations 501 is designed to add functionality to the system in being able to determine the presence of more than one intoxicant. Here when the results of the pharmacodynamic sequences are indeterminate or negative 512, despite a positive toxicology finding 513, further analysis is performed. First the dynamic psychopharmacologic test sequence data is tagged and associated with the findings of toxicology in the database 514. The database is searched for other similar findings involving one or more of the drugs found in combination, as well as negative cases for impairment 515. The gathered data is cross analyzed for classifiers and distinguishing features from the controls which are consistent with the drug combination 516. These features are extracted and a new set of rules, parameters, and/or output conditions are assigned to the computation layer(s) of the machine learning model to detect the combination 517. Finally, the new set of rules are tested against the existing database and further optimized to ensure the differences between the test and train data remain within an acceptable margin of error for all test conditions.

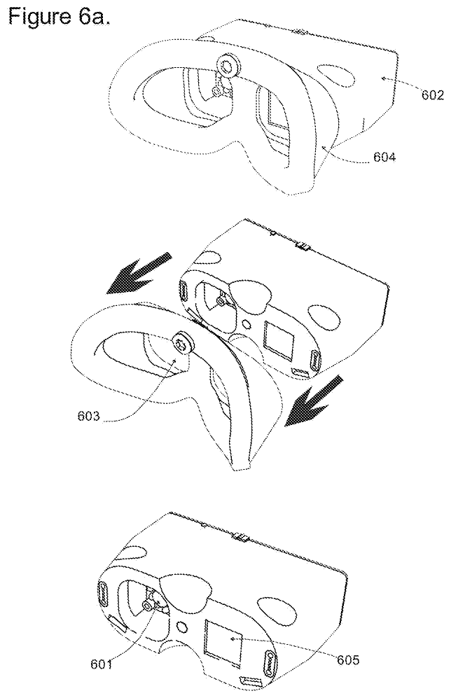

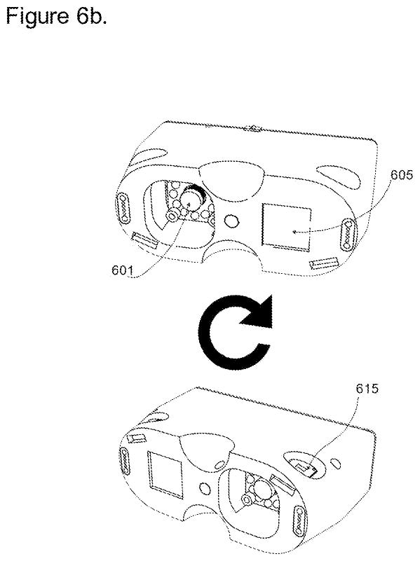

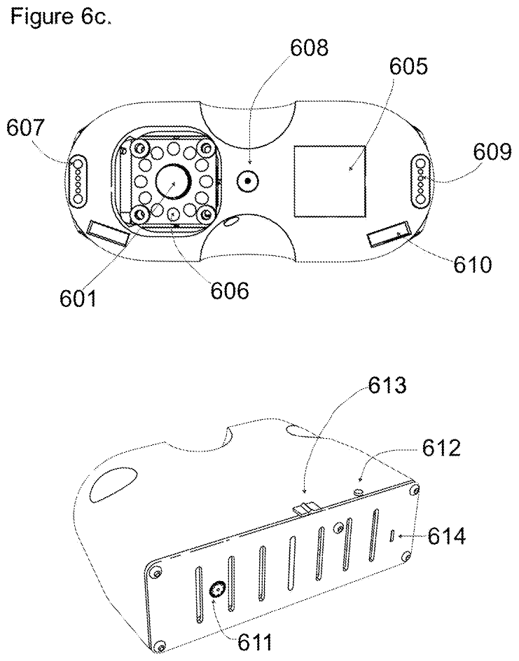

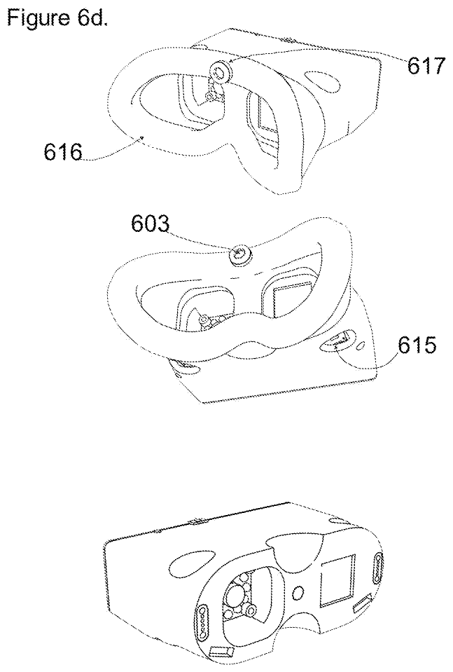

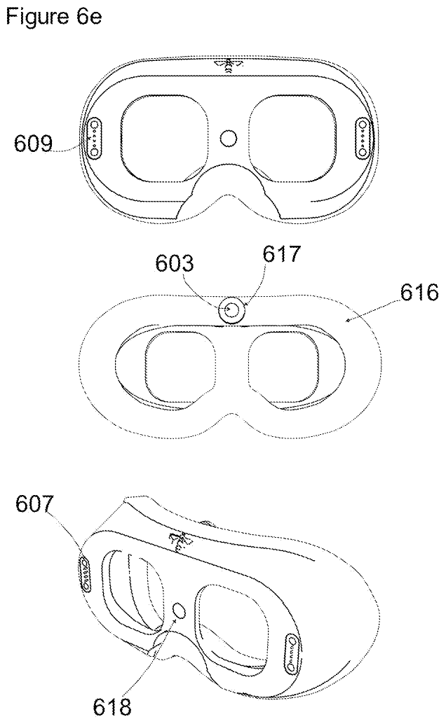

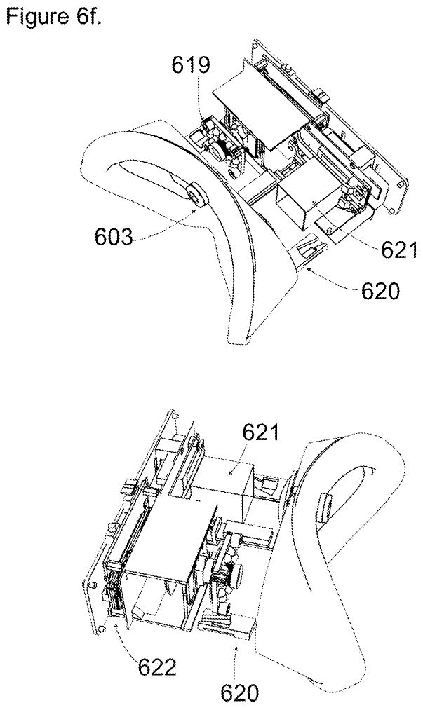

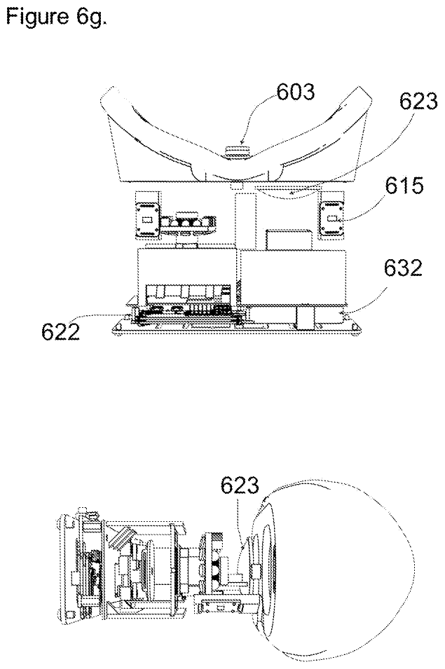

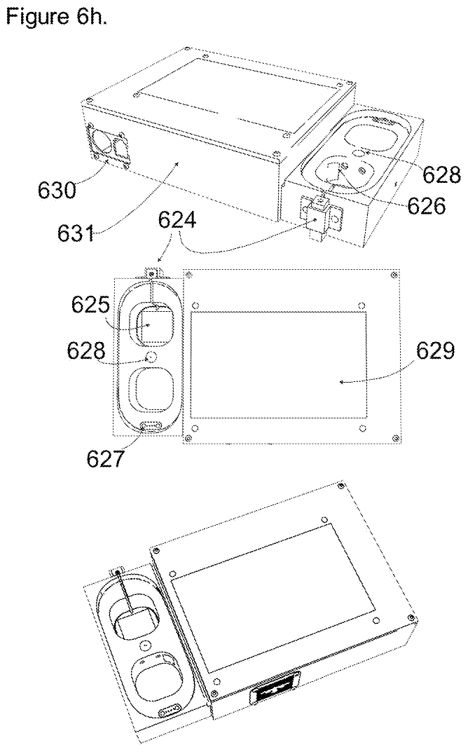

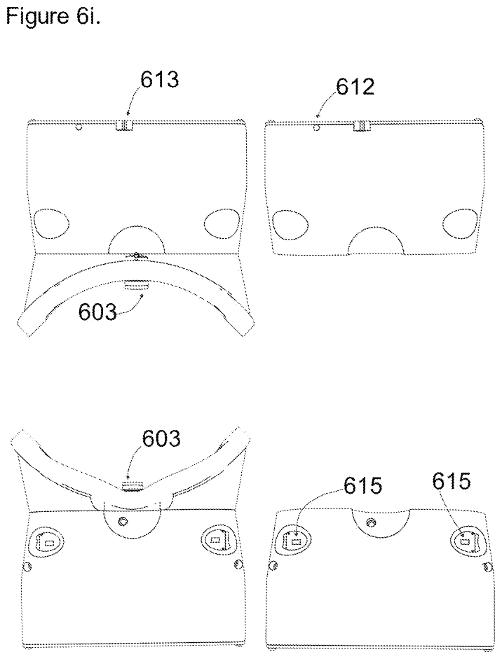

FIG. 6A presents an example eye scanner 101 for use in the workflow described in FIGS. 1A and 1B. The device is a hand-held pupillometry and vital sign measuring device 602 which can be used to gather the pharmacodynamic data inputs. It consists of a single-camera 601 with a magnetically detachable mask 604, with an embedded pulse sensor 603. In some embodiments the pulse sensor 603 may perform photoplethysomography measurements to determine pulse. In other embodiments this is determined through a dry electrode electrocardiography sensor, with other electrodes positioned around the face mask (not imaged). Though the combinatory use of illumination in the visible and IR spectra by LEDs or other light source 606 sensed by the camera 601 and other visual input from the included display 605, the eye scanning device measure the eye's reaction to various visual input stimuli.

FIG. 6B describes how the eye scanner 602 can be oriented in such a way that allows for the measurement of both eyes. The device can be flipped from its regular position which measures the left eye, so that the camera 601 is viewing the test subject's right eye. The modularity of the facemask 604 allows this to function as the test administrator can magnetically couple the facemask to the eye scanner device 602 after it has been flipped.

FIG. 6C details the overview of system components. In this embodiment, the image sensor 601 detects in the VIS-NIR wavelength range and is surrounded by a ring of LEDs 606 which consist of both infrared and visible light emitting diodes. Here, the infrared diodes provide the background illumination for the image sensor, while the visible light diodes provide the stimulation for pupillary light response. In addition, the display 605 provides further options for stimulation and test sequences. For example, in the OKN tests for the confirmatory sequences 123 described earlier, a full screen grated bar pattern can be displayed on the screen which moves left and right. In another embodiment, attention can be measured instructing a test subject to fixate gaze on a large central image while a flickering patch at some frequency (e.g., 2 Hz) is displayed in the periphery. Attention by pupillary position and size can be used to determine the differences between divided, covert attention (if subject is monitoring the periphery) and over attention (if subject is monitoring the central image).

To connect the eye scanner 602 to vital sign measures on the detachable facemask 604, magnetic connectors are illustrated 607, 608. These allow for the consistent alignment of the mask onto the eye scanner while also functioning as a relay for power and data. Analog signals generated by the sensor 603 or other sensors positioned on the modular mask 604 are transmitted over the pins 609 on the left and right magnetic connectors 607. In addition, the pins 609 and transmit power to the sensors positioned on the mask 604. The device can sense which eye is being measured by determining which magnetic connector is transmitting a data signal from the mask's sensor, because the vertical orientation (data, power, ground) of the pins 609 is fixed. The central connector 607 further serves as the input for charging the device's onboard battery when docked at the base station shown in FIG. 61. The slots 610 allow for sensor modules positioned under the test subject's fingers to be swapped out for other sensors.

Further illustrated in FIG. 6C. is the front of the device which consists of a front facing camera 611 and a standard I/O interface. The camera 611 may in some embodiments be used to label the test data with the test subject's identity (e.g., with a driver's license). In further embodiments it can be used to monitor the activity of the test administrator and surrounding test environment for review as needed. The button 612 allows for the reset of the device, while the switch 613 allows the test operator to turn the system on and off. The LED 614 provides an indication of device status to the test administrator, informing when a reset may be necessary.

FIG. 6D demonstrates an example positioning of multiple pulse sensors 603 and 615 on the eye scanner. Here, a pulse sensor 603 is placed directly over the supraorbital artery in the forehead, while the backing material 617 ensures a consistent pressure is applied by the forehead against the sensor. This material 617 varies in hardness and/or density from the surrounding softer foam 616 of the detachable facemask 604. Additional sensors 615 are positioned under the test subject's fingers for comparison study. Comparing the wave forms between the three sensors can be used to determine pulse transit time. Monitoring changes in pulse transit time has been previously described as a surrogate for detecting changes in systolic blood pressure.

FIG. 6E illustrates additional views of the detachable facemask 604. Notably the pins 609 can transfer analog data from the mask back to the eye scanner, allowing for further customization of the mask itself. In one such embodiment, the mask can house multiple electrocardiogram sensors positioned along the interfacing foam 617 to add additional signal resolution. In another embodiment the mask can house fNIRs sensors for brain activity positioned above the brow of the test subject. In another embodiment the mask can house electrical sensors positioned around the eye for a portable electroretinography device which monitors responses to different wavelength stimulation from the display 605 and LEDs 606 on the eye scanner.

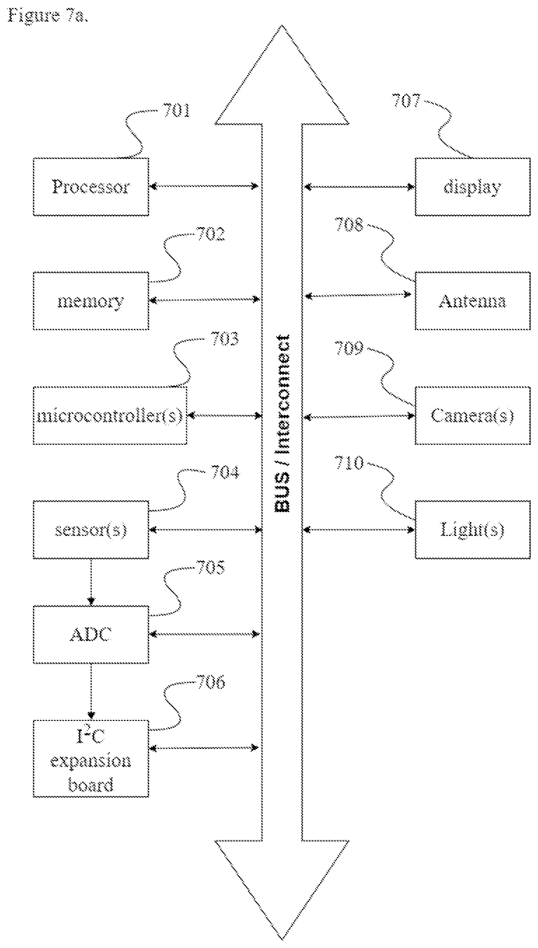

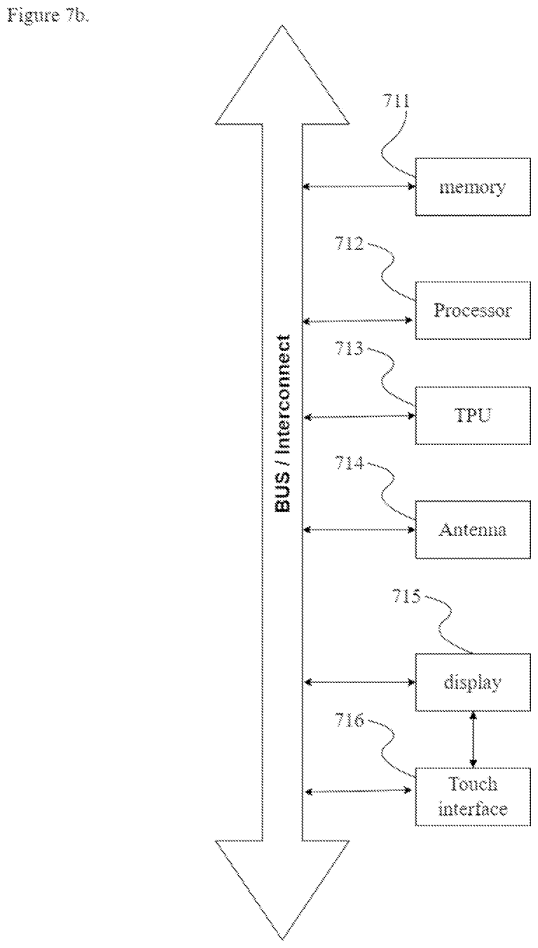

FIG. 6F illustrates the internal components of the eye scanner. The primary compute board 622 for the eye scanner hosts a computer processing unit, volatile memory, an analog to digital converter, power management, networking interface such as a Wi-Fi antennae or Bluetooth, nonvolatile data storage (e.g. SD card or onboard solid state drive), data interface (e.g. USB), and optionally a hardware accelerator (graphics processing unit, tensor processing unit, field programmable gate array, application specific integrated circuit, etc.) as detailed further in FIG. 7. In addition, a mount 619 hosts the LEDs 606 configured radially around the camera module 605. To ensure excess light from outside the eye scanner or generated by onboard LEDs does not interfere with the test sequence, the light isolation component 621 is positioned directly over the display 605. This light isolation component 621 is a hollow column with light blocking walls such that the eye can receive light stimulation exclusively from the display 605. Finally, the sleds 620 host the finger pulse sensors 615 and allow for further modularity. A test administrator can access the sled 620 through the slots 610, swap out the sensors 615 attached to the sled 620, and re-insert them into the eye scanner again through the slots 610.

FIG. 6G further illustrates the internal components of the eye scanner through different views (top and profile) of the device. In particular, an asymmetric biconvex lens 623 is shown positioned between the test subject's eye and the display 605 with the light isolation component 621. This lens is tailored to the focal distance to the display, allowing the images displayed to be magnified and reducing eye strain on the user. In addition, a rechargeable power source 632 (e.g., a Lithium Ion Battery) is included and connected to the power management of the primary compute board 622, allowing for portability.

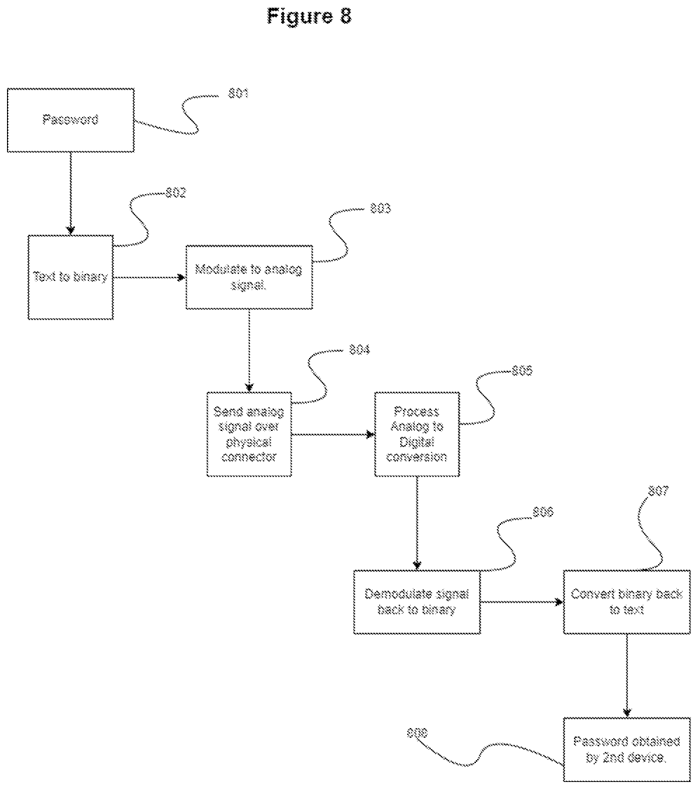

FIG. 6H demonstrates a base station for the eye scanner and interface for the test administrator. When finished with a test sequence the magnetically coupled mask 604 is separated from the eye scanner 602. The eye scanner 602 itself is placed directly in the dock 626, assisted by the magnetic guides 627 and 628. Once docked, the eye scanner begins a calibration sequence. Here, a calibration pattern 625 (e.g., a checkerboard of fixed size) is printed on the dock in the direct line of sight of the onboard camera 601. A stage 624 moves the printed calibration pattern in the vertical direction along the focal length of the camera. The system calibrates its interpretation of measurements in pixels to the known measurements of the pattern 625 based upon the vertical position of the pattern. The power connector 628 charges the internal battery 632 on the eye scanner. Following the eye scanner 602 docking, the dock will further provide network credentials to the eye scanner through a secure analog signal transmitted via the magnetic connector 627, so that data can be transmitted from the peripheral device to the base station. This is detailed in the workflow diagram FIG. 8.

The test results are displayed on the touchscreen 629, which allows the test operator to interact with and access the data. In addition, this interface allows the test operator to initiate the test sequences and respond to input inquires by the system itself (for example, 113 and 128). The touchscreen is housed in the base station 631 which hosts the systems that process the pharmacodynamic and pharmacokinetic data collected by all sensor systems and interprets the probability of impairment. The base station 631 may in some embodiments periodically upload test results to a deep learning system hosted in a server or cloud environment through an ethernet connection 630 or wirelessly. This deep learning system would then provide optimized test parameters, rules, system weightings, and other variables to improve the effectiveness of the system to determine the probability of impairment. In addition, the port 630 allows for additional sensors to be installed on the base station as need, for example a toxicology device.

FIG. 7A. Outlines the components attached to the bus of the eye scanner 602. The processor 701 and memory 702 used is from a series of small single board computers for computation 622. In some embodiments, this is prefabricated board such as the raspberry pi, used to handle communications and onboard computation throughout the device. In some embodiments, this is a custom made-to-order SBC. In some embodiments, this SBC 622 will also include the antenna 708 for communications. The front facing camera 611, the board for the white light and IR LEDs 611, and the display 605 are all handled by a separate MCU (microcontroller unit) that communicates with the main SBC 703. In some embodiments, these are low-cost system-on-a-chip microcontrollers. In some embodiments, they are prefabricated chips such as the ESP-32. For the sensor end of this device 602, the main SBC 622 handles all the inputs from the main camera 601, the finger sensors 615, the ADC 705, as well as the I²C expansion board 706 that feeds the ADC 705 into the system, which originally takes input from the forehead sensor 603.

FIG. 7B outlines the components attached to the bus of the base station 631 and dock 626. For the base station, handling the main processing 712 and management of the input 711 from the eye scanner, another more powerful SBC is used. In some embodiments, this is a raspberry pi4. In some embodiments, the base station 631 houses a portable mini-form desktop computer. In some embodiments, some portable computer is used for data processing. As well, an additional machine learning ASIC may be used 713 to assist in any machine learning type workload. In some embodiments, this can be a coral edge TPU 713 or some other custom Application-specific integrated circuit. In some embodiments, this can be a FPGA, GPU or other hardware accelerator 713. Further embodiments may make use of any combination of these hardware accelerators described. For the base station, the user input and displayed results are both a function of the attached touchscreen 715 716. Lastly, the base station has a communication antenna for transmitting and receiving data from the eye scanner 602 during test sequences, as well as transmitting and receiving data from a remote database 119 for optimizing one or more of the processors and/or machine learning instances. For this disclosure, antennae may include wireless communication protocol including Bluetooth and Wi-Fi, but may also include wired communications such as ethernet. Any combination of these communication methods may be employed.

FIG. 8 describes the secure hand-off of network credentials from the base station 631 to the eye scanner 602 by way of the dock's magnetic pin connectors 627 interfacing with the eye scanner's magnetic pin connectors 609, 804. Pictured, the starting device 801 takes the password, or wireless credentials, and converts that string of characters to binary code 802. That morse code is then modulated to an analog signal. 803 In some embodiments, this analog signal comes in the form of a waveform audio file (.wav). In some embodiments this is achieved by a simple carrier wave. In some embodiment this is a simple audio file. That analog signal is then expressed over the magnetic connectors 609, 627 when docked with the base station and read by the analog-to-digital converter 804. The second device then records that analog signal, and processes to back into a digital signal. 805 That signal is then converted back to binary morse code 806 and then further converted back into a string of usable text. 807. Then the second device, in this case the eye scanner 602 is now free to use the password obtained through the credential pulling handshake procedure. 808.

FIG. 9A describes a generalized overview of the toxicology assay process 109. First an onboard identifier of the cartridge associated with specifications along with a unique string or hash is read by the device reader 901. In some embodiments the data is stored in a radio-frequency identification (RFID), nearfield communication (NFC), and/or Bluetooth low energy (BLE) format located on the test cartridge. In other embodiments, only the string or hash is kept on the chip in a barcode or quick response code. This is imaged by an optical sensor in the device reader and interpreted as a string or hash associated with the cartridge. In one embodiment the specifications of each cartridge are stored in the base station 631 and updated periodically by a remote server. In another embodiment the hash is sent directly to a server or cloud hosting the database and the associated cartridge specifications are sent back to the base station 631 and/or cartridge reader.

Following the identification of the cartridge and the test stored on it, the reader receives the sample 902. In some embodiments, the collected sample is inserted into the cartridge and secured with a locking or pressure-fit mechanism. After this it interacts with the fluidic system described herein. In other embodiments, the sample is inserted into the cartridge first before placing the loaded cartridge into the reader. The mechanical or pneumatic force provided by the locking or pressing mechanism of the sample into the cartridge and/or the cartridge into the reader releases the sample and prepares it 903 for interaction with the biosensors. In some embodiments this includes the release of a buffer solution from hermetically sealed capsules and mixing the sample on the cartridge. Mixing can be based upon a combination of sonication, centrifugation, magnetic bead assisted, and/or passive (based on flow). In some embodiments, the use of on-chip filtering strategies is employed including, but not limited to the use of H-filters, size exclusion membranes, filter paper and centrifugation.

Next the prepared sample is released for interaction with the biosensor and detected by the optical sensor 904 as further illustrated in FIG. 11. The optical signal is measured and interpreted as a series of ligand concentrations FIG. 3A and those concentrations are further interpreted as part of the overall probability of impairment as described in FIG. 2. The results of the analysis are shown 905 on the base station's display 629. Finally test results are stored on the cartridge itself in the format of RFID, NFC, and/or BLE as well as the base station for further training 906.

FIG. 9B describes a more specific workflow and embodiment of FIG. 9A which allows for the use of a dehydrated cell-based biosensor, and optionally allows for the storage of the collected sample matrix 907 after it has been pre-filtered. First the cartridge is scanned by the reader and the device is unlocked 901. Then the sample is pre-filtered for larger contaminants 908 by, in some embodiments, the use of paper filters or other cellulose based products. The pre-filtered sample passes into a mixing well, and at the same time the pneumatic pressure or mechanical force provided by inserting the sample into the cartridge and/or cartridge into the reader triggers the release of the buffer from the hermetically sealed capsule into a mixing well 910. As above, mixing can be based upon one or more of sonication, centrifugation, magnetic bead assisted, and/or passive (based on flow). The fluidic system in some embodiments includes a piezoelectric sensor to measure the viscosity of the solution containing the sample and buffer. This sensor would inform the system when to stop mixing.

Embodiments which make use of centrifugation on the cartridge to homogenize the sample with buffer, for example with a magnetic bead, may not require another filtering step 911, instead the well walls are designed to trap smaller contaminants. In other embodiments, a filter for smaller contaminants is applied to the sample 911 including, but not limited to H-filters, size exclusion membranes, and filter paper will smaller pores than the pre-filter. Some embodiments may make use of any combination of the described filtering strategies. In embodiments where confirmatory analysis is required, a portion of the sample can be diverted after the mixing process into a storage well for further analysis 907.

Once the sample has been filtered 911, it is further prepared for interaction with a dehydrated cell-based biosensor assay. The next step is to warm the sample 912 to the ideal physiological temperature of the biosensor assay. In one embodiment of the system, using S. cerevisiae, the cells need to ideally be kept at 30° C., thus the filtered sample will be warmed to this temperature. To achieve this a thermocouple may be placed inside the chamber to instruct the control system when to stop the heating process. In some embodiments, the system will simultaneously look to remove excess air pockets from the collected sample through methods including sonication and/or vacuum pressure, allowing dissolved gas to escape through a membrane leading to the pneumatic line. In other embodiments the heated sample passes through a mechanical bubble trap, which is equipped with a membrane to allow the gas to escape.

The homogenized, warmed, and de-bubbled sample is finally pulled into the sensor well(s) for interaction with the housed eukaryotic cell assay 913. A pneumatic gate will allow for the vacuum pressure derived from manual force or a pump system to be applied to the sensor well, thus opening the valve separating the heating/de-bubbling chamber from the sensor well. In some embodiments the pneumatic gate can be signaled by the thermocouple to open the valve when the sample reaches the desired temperature.

The dehydrated cell-based biosensors are rehydrated in the presence of the homogenized and warmed sample 913. The interaction well will contain a metabolic sensor for cell activity, which upon reaching a pre-determined threshold, triggers the optical detection of the well 914. Typically, metabolic sensors may look to detect ethanol, carbon dioxide, air pressure, and/or calorimetry. In the embodiments where carbon dioxide and/or air pressure is used, the pneumatic gate linked to the well with a hydrophobic membrane barrier will house the sensor. Ethanol may be detected with a fuel cell. Finally, the optical sensor measures the wells' luminescent or fluorescent signal 905 and the results of the analysis are stored 906 as previously described.

FIG. 9C describes the same initial steps through sample incubation as FIG. 9B 901, 907-913, but allows for the integration of a wash consisting of ethanol, methanol, or dimethylsulfoxide. In one embodiment where an external substrate is required, for example with a luminescent reporter system, the substrate may be suspended in the wash solution or in another hermetically sealed capsule. Some additional shielding from degradation (i.e., from light exposure during storage) may be required for the capsule housing the substrate, which is typically volatile. In this embodiment, the substrate would be released 921 along with the wash 915 and pulled into the wash well 916. In some embodiments, the wash and the substrate suspension may be mixed, if required, through a one or more of sonication, centrifugation, magnetic bead assisted, and/or passive (based on flow). In some embodiments, where the sample matrix is heated 912, the wash and/or substrate suspension may also be heated with to the same temperature with a thermocouple in the wash well prior to interaction with the biosensor well. Next the wash and/or substrate suspension is pulled into the sample well triggered by a pneumatic gate, which also opens the valve in the channel connecting the wash and sensor wells 917. In some embodiments the pneumatic gate may be signaled by the thermocouple to open the valve when the sample reaches the desired temperature. Following this step, the pneumatic gate can reverse the pressure and pull the mixture of the sample, wash solution and/or substrate back into the wash well 918 and closing the valve. This may be repeated 919 as many times as is experimentally determined to be optimal. When the washing step completes, the resultant solution is pulled back into the wash well and the valve is closed one last time 920. A metabolic trigger signals the optical sensor to pick up the signal from well as described above 914, the results are interpreted and displayed on the base station 905, and finally stored for further analysis and optimization 906.

FIG. 9D describes the integration of cytometry methods into the cartridge in order to improve the sensitivity of the system. The system follows the same initial steps through sample incubation as FIG. 9B 901,907-912. However, in parallel with the small particle filtration 911, the system will use the same pneumatic or mechanical force by which the filtration is carried out to also release the substrate suspension from the hermetically sealed capsule 922, if required by the biosensor. In embodiments where multiple biosensor interactions will take place in the same channel a fluorophore may be included in the capsule containing the substrate and suspension liquid. This would allow for each interaction (e.g., different receptors or controls) to have a tag with a specific wavelength range defined by the fluorophore present.

The substrate and optional fluorophore would be held in the prep well and in some embodiments warmed using a thermocouple to the same temperature as the sample matrix in step 912 and/or de-bubbled 923 using sonication, mechanical bubble trapping and/or vacuum pressure. The prepped sample matrix, substrate, and optional fluorophore would be released into the incubation well housing the biosensors 924 triggered by the opening of valves between the incubation well and each of the preparation wells by a pneumatic gate. In some embodiments the pneumatic gate may be signaled by the thermocouple to open the valves when the sample reaches the desired temperature. The biosensor incubates in the presence of sample, substrate, and option fluorophore 925.

Upon the metabolic trigger which may be a threshold of ethanol, carbon dioxide, air pressure, and/or temperature, the hydrophobic liquid is released into the droplet generator. In some embodiments a pneumatic gate triggered by the metabolic sensor controls a valve separating the hydrophobic liquid from the droplet generator. Subsequently, the incubated biosensor suspended in the sample matrix, substrate, and optional fluorophore is also released into the droplet generator 927. The flow rates of each may be adjusted by the pneumatic gates to ensure an even distribution of yeast cells among individual droplets. In some embodiments yeast cells incubated in a control matrix, substrate, and distinct fluorophore from the active sample are passed through the droplet generator as well 928. Next these droplets are sorted by the presence of an optical signal into positive and negative selection wells 929. Finally, an optical detector measures the resultant cell counts in each well 930. In embodiments which make use of fluorophore tags, the optical detector may be spectrally filtered to the emission wavelengths. In addition an excitation light source positioned to illuminate the selection wells may also be spectrally filtered as required.

FIG. 10A illustrates a schematic for a cartridge which can perform the sample processing steps described in FIG. 9B. First the sample is inserted into the cartridge at the inlet 1001. In some embodiments the mechanical force from inserting the sample (e.g., from a swab) and/or locking it in place is transferred pneumatically to the rest of the system via 1007. In other embodiments the force can be provided from inserting the cartridge with the same into the reader, a manual pressure-driven pump (e.g., finger pump), a vacuum pump, and/or with a mechanical pumping system (e.g. syringe, pressure, peristaltic). In any of these embodiments, a hydrophobic membrane or ‘stop’ 1008 is positioned between the liquid lines and the pneumatic lines. The pneumatic lines may carry a high pressure or a vacuum environment, wherein a vacuum would diffuse air from the liquid lines into the pneumatic lines and vice versa for high pressure. However, this membrane would prevent and liquids from leaking into the pneumatics. In one embodiment, the membrane is made from PDMS. The flow rate for micropumping via pneumatic lines considered constant as a result of Fick's law. In some embodiments the flow through any of the modules on the cartridge is assisted by means of passive capillary force.

Next the pre-filtering of the sample takes place by means of a cellulose-based product 1002 which removes larger contaminants before interacting with microfluidic channels. Concurrently the pressure difference in the pneumatic line 1007 and the hermetic capsule 1003 causes the release of the contained buffer solution. Both the pre-filtered sample and buffer enter the mixing well 1004. Mixing can be based upon one or more of sonication, centrifugation, magnetic bead assisted, and/or passive (based on flow). The well 1004 in some embodiments includes a piezoelectric sensor to measure the viscosity of the solution containing the sample and buffer. This sensor would inform the system when to stop mixing.

The pneumatic force 1007 pulls on the membrane or h-filter 1006 drawing the mixed sample through and eliminating smaller contaminants and opening a valve 1009. Next the sample is drawn into the heating and de-bubbling chamber 1010 and prepared for interaction with the cell-based biosensor assay. Once enough filtered sample is prepared, filling the heating and de-bubbling chamber, the valve is closed due to the normalization of pressure and the next step is triggered. Here the sample temperature is adjusted to the ideal physiological temperature for the biosensor assay. In embodiments which make use of yeast-based biosensors this is typically 30° C. Dissolved gas is pulled from the sample by means of a vacuum in the pneumatic line. When the ideal temperature is achieved, the valve 1011 is opened and the prepared sample interfaces with the cell-based-biosensor in a channel or well 1012. In some embodiments the channel or well 1012 employs mixing strategies based upon one or more of sonication, centrifugation, magnetic bead assisted, and/or passive (based on flow). Excess liquid is managed by the overflow chambers 1005. In one embodiment the pneumatic overflow module 1013 is equipped with a sensor (e.g., for CO₂) to detect when the cell based biosensor is metabolically active, triggering the optical detection of the well or channel.

FIG. 10B describes a method of adapting a wash into the cartridge layout described in FIG. 10A and carries out the handling steps described in FIG. 9C. All the fluid handling is the same until the sample arrives in the sensor wells or channel 1012. Here the wash 1016 is released into the wash well 1017 and prepared for interaction with the sample and biosensor. In some embodiments, this involves heating and de-bubbling the wash in a similar manner as is performed in 1010. Once the wash is prepared, the pneumatic gate 1014 will periodically oscillate the pressure in the pneumatic line attached to the sensor well(s) or channel(s) and the wash well(s). This ensures an even distribution of analyte is exposed to the biosensors. When the pneumatic line 1007 pulls the wash back into the well 1017, the valve 1015 is shut. The wash is held in the well during the optical detection process.

FIG. 10C describes a method of adapting an additional substrate into the wash in embodiments where the substrate needs to be stored independently of the wash solution. This may be required when the substrate is particularly volatile and sensitive to degradation such as those derived from luciferin, furimazine, or coelenterazine. Here the substrate is housed in a hermetic capsule 1018 which in some embodiments may be adapted to block out ambient light. When the pneumatic gate 1014 is initially triggered, the system will simultaneously extract the contents of both the substrate suspension and the wash solution. The wash well 1017 may also be adapted to mix the substrate suspension and the wash solution by previously described means, in particular sonication may be desirable as it can both facilitate mixing and de-bubbling.

FIG. 10D describes an adaptation of the cartridge design to accommodate a cytometry-based approach to improving system performance and sensitivity, carrying out the handling steps described in FIG. 9D. All of the fluid handling leading up to the sample warming and de-bubbling in 1010 remains consistent with other Figures and embodiments. However, concurrently with the filtering step in the module 1006, the substrate and optional fluorophore is released from hermetic capsule 1019 into the prep well 1020. Both the prep well 1020 and the warming & de-bubbling 1010 turn on around the same time (when the respective liquid enters the well). The sample matrix and substrate with optional fluorophore are both heated to the same temperature and simultaneously released by valves 1011 and 1021 into the incubation well 1031. When a metabolic trigger or threshold is reached, detected as previously described by the pneumatic gate 1014 or a sensor in the incubation well 1031, a valve 1024 opens triggered by the pneumatic gate 1022 and releases the hydrophobic liquid 1023 into the droplet generator 1026. Subsequently the cells are released from the incubation well 1031 into the droplet generator 1026 as a result of the valve 1025 opening. There are numerous embodiments of configurations which would allow for yeast cells to be trapped in an aqueous droplet surrounded by a hydrophobic carrier, including but not limited to flow-focusing designs, step emulsification, co-flow, and T-junction. The suspended droplets of biosensing cells, sample matrix, substrate, and optional fluorophore as sorted by the cell sorter 1027. In a typical configuration of fluorescent cell sorting, an excitation laser is passed through the fluidic channel, and charged plates are arranged along the channel fork leading to the positive collection well 1029 and negative collection well 1030. Cells with a fluorescent signal carry a positive charge while those without carry a negative charge. In one embodiment an optical sensor is introduced along the channel following excitation and counts the cells. In another embodiment the cells in the resultant wells 1029 and 1030 are counted at the end of the process by an optical sensor and interpretation algorithm.

FIG. 11 describes a generalized overview of the image sensor's function on the cartridge reader following the trigger of the metabolic signal 914, 1101. The metabolic signal is produced with the cell-based biosensors have been rehydrated in the sample matrix and are metabolically active, capable of producing an optically detectable signal. In embodiments, where the biosensors utilize a fluorescent reporter, an excitation light source is then powered 1102. In embodiments where multiple fluorescent tags are present in the same detection well or channel, the light may be spectrally filtered to the fluorescent tags' excitation wavelength. This may be accomplished in some embodiments through a stage which moves color filters over a white light emitting laser. Next the emission wavelength from the channel is sampled by the optical sensor 1103. Each well is sampled 1104 for both intensity and shift in wavelength and the values for each are plotted over time 1105. The plots are cross compared 1106 as described in FIG. 3 and the process continues until all wells have been sampled 1107. Finally, ligand values are determined 1108 and the report is sent off 1109 to the test administrator's display 629 and for further training the model 119.

A summary of the inventions disclosed herein is provided in this and the following paragraphs. A system and method are disclosed for determining the presence of neurological impairment by one or more intoxicants in a subject. An instrument measures the state of the autonomic nervous system by detecting changes in pupillometric and vital sign measurements in response to programmed stimuli. This is achieved by an instrument for measuring the concentration of metabolite(s) of an intoxicant and/or endogenous molecule(s) in a sample matrix which are altered by the presence of the intoxicant metabolite(s) and a processing unit for analyzing the collected data by one or more instruments and determining the probability of impairment. The sample matrix may be processed by a cartridge system which accepts a sample, filters it, optionally stores it, and prepares it for interaction with a biosensing assay housed on a micro- or milli-fluidic chip. The system my include a sample collection tool which gathers the biological matrix of interest and, in the case of saliva, pre-filters larger particles from the sample before interacting with the rest of the cartridge. An on-chip mixer may be provided for interacting the sample with a buffer solution stored on chip. The system my use on-chip thermal regulation to maintain ideal temperatures for the rehydration of a host cell. In some embodiments, this may be applied to the sample matrix, buffer, wash, and/or substrate suspension prior to interaction with the assay.

Microfluidic well(s) housing a dehydrated eukaryotic model organism may be provided for interaction with a filtered and prepared sample. These well(s) may also include mixing, sonication, or other means of homogenizing the analyte, biosensor, and/or substrate as needed. The sample may be interacted with the biosensing assay, and the optical data that is generated may transmitted to a detector.

The cartridge system of may contain breakable hermetical capsules that store their contents (e.g., buffer, reference samples) until undergoing a mechanically (or chemically) triggered burst-release.

One or more components of the cartridge may be 3D printed, and where the material is not natively bio-compatible, subsequently coated (e.g., vapor, spin, grafting, and/or sputter) to prevent biofouling. One or more components of the cartridge may also be produced through commercially available means including soft lithography and/or micro-milling.

The cartridge may contain biosensing cells which function as a model organism stored onboard the cartridge. In some embodiments, the chosen model organism may express significant amounts of trehalose to tolerate the stress of dehydration and rehydration. The chosen model organism will express endogenous G-protein coupled receptors which can be genetically edited and replaced with a receptor for (a) target analyte(s). Cells of the species Saccharomyces cerevisiae may be subjected to previously described methods to maintain a dehydrated active-dry or instant state for stable storage at room temperature for extended periods and off-the-shelf use.

Where the collected sample is saliva, the cartridge system may utilize a starch-based (or similar polysaccharide-based) glue to adhere the dehydrated active-dry or instant cells to their respective wells. Upon rehydration, the cells will use the enzymatically cleaved disaccharides as metabolic fuel.

The mentioned microfluidic well(s) may be equipped with one or more of the following sensors which upon achieving an experimentally described threshold, triggers the capture of the optical reporter signal in the biosensor. This may comprise: calorimetry with an onboard thermal sensing element; air Pressure in the pneumatic overflow/gate with or without an onboard CO2 sensor; and/or ethanol sensor by means of a fuel cell.

The microfluidic well(s) may function to relay the optical signal output by a live cell assay to a pickup unit. The cartridge may contain micro lens caps for every individual model organism well or channel and/or reflective coatings in the well housing the model organism to improve the fidelity of the signal. In the case of a fluorescent optical reporter, this will comprise an excitation light source and a spectral filter allowing to separate the signal between the excitation and emission wavelengths. In the case of luminescence, this will comprise a photomultiplication method and/or a modified substrate for an expressed luminescent enzyme reporter.

Also disclosed is a method of generating assays specific for the detection of an analyte or multiple analytes, utilizing one or more of the following processes: determining an analyte's concentration by comparing between mutants of the same receptor, each with different binding affinities for that target analyte; in one embodiment these mutants may be generated through directed evolution of the receptor in the presence of that target analyte and screening for variance in binding affinities. In a further embodiment mutagenesis is induced through polymerase chain reaction in the receptor's experimentally determined receptor binding domains to achieve greater variance in affinity. an analyte's concentration may be determined by comparing the signal generated by two or more biosensors expressing distinct receptors for the same analyte. In the case of THC, this may consist of previously noted receptors including, but not limited to: CB1, CB2, GPR18, and GPR55.

Also disclosed is a method of analyzing the multiplexed assay data described by comparing values between wells and accounting for the anticipated binding affinity ratio of analyzed wells. Deviance from anticipated signal ratio is resolved by the combinatorial analysis of other well ratios. In some embodiments the combinatorial analysis is resolved by computational layer(s), whereby the computational layer(s) is/are determined by training using input receptor sets and reference analysis. The described method may be useful for determining ligand values in the absence of controls.

A method is disclosed of improving the sensitivity of the system and reduce signal noise through the use active control or a lower-affinity ligand with inverse function to the analyte for use in a typical displacement assay. These controls may be stored on the cartridge itself as described in claim. One embodiment of the cartridge system may include a fluidic gradient generator to provide varying concentrations of active control and/or a weakly binding ligand for a displacement assay. An embodiment of this system may utilize fluorophore-tagged ligands in a displacement assay and measure the change in optical signal following exposure to the sample matrix.

A method is disclosed for improving the sensitivity of the system and reduce signal noise by processing the model organism through flow cytometry on the cartridge, wherein cells are sorted by the presence of a fluorescent or luminescent signal following exposure to the filtered sample matrix and cell counts are associated with ligand concentration by a processor. In one embodiment each cell is isolated in a single droplet containing the sample matrix and buffer and these droplets are suspended in a hydrophobic liquid before passing through the cytometer. The refractive index of the droplet and suspension liquids may be selected in such a way as to create a micro-optofluidic lens, enhancing signal sensitivity further. In embodiments where a substrate is required, such as a luminescent reporter system requiring luciferin, furimazine, or coelenterazine, the substrate will be suspended in the droplet. In embodiments which make use of controls, the cells will be placed in droplets containing the control samples and sorted in parallel channels against the cells containing the sample.

In addition, a method is disclosed of running cell sorting assays in the same channel by tagging each droplet with a fluorophore or other molecule which alters the wavelength of the optical signal produced by the droplet. The cell counts for each wavelength range would be compared against each other using spectrally filtered optical sensors. In embodiments which make use of multiple receptors, each distinct receptor would be assigned a tag of a pre-specified wavelength. In embodiments which make use of controls, the control droplets would be assigned a distinct tag from the active sample.

Also disclosed herein is a system for the detection of the state of the autonomic nervous system, where an instrument automates the pupillometric measurements that are typically manually performed by police Drug Recognition Experts, namely: (1) lack of convergence, (2) pupillary hippus, (3) rebound dilation, (4) vertical gaze nystagmus, and/or (5) horizontal gaze nystagmus. The instrument may utilize a single camera directed towards the eye which can detect in the VIS-NIR wavelength range. The instrument may simulate a dark-room environment by shielding the eyes and internal optical components from external light. The instrument may utilize a series of IR-LEDs for consistent illumination throughout the test sequence as well as visible light LEDs or a small screen for stimulating the pupillary response. The visible light stimuli may move to determine accuracy and latency for positional tracking.

The described instrument may calibrate its real-world measurements when docked by analyzing a pre-defined pattern printed on the interior of the dock within the focal distance of the camera.

Also disclosed is a system for the detection of the state of the autonomic nervous system where the system may run test sequences of various visible or auditory stimuli and gather data on how a subject's vital signs respond. This may include some combination of (1) heart rate, (2) respiratory rate, (3) blood pressure, (4) temperature, (5) pupillary size, and/or (6) pupillary position. The vital signs can be analyzed alongside user feedback (e.g., auditory or through a button/switch) which is measured for accuracy and latency thus providing more clarity for the neurological status of the subject. The measurements may be gathered by multiple photoplethysmography (PPG) and/or electrocardiogram sensors placed on the finger grips as well as the facial contact points of the eye scanner itself, such as the middle of the forehead (supratrochlear or supraorbital arteries), adjacent to the ears (superficial temporal artery) or on the sides of the nose (angular or dorsal nasal arteries). The measurement of vital signs may be enhanced or replaced with a multi-spectral or hyperspectral camera array where aggregate image data will be analyzed in the 500-660 nm spectra for hemoglobin oxygenation and spatiotemporally mapped. The measurements may be measured in a timeseries of 2D snapshots in multiple spectral bands: i.e., non-scanning hyperspectral imaging or a multi-spectral camera array capturing images in parallel. The measurement may be a series of spatio-spectral scans made with a hyperspectral imager.

The measurement of vital signs may be enhanced by mapping hemoglobin against temperature data gathered from an IR sensor for higher confidence in the model, while optimizing performance of the model by selecting regions of interest defined by gathered temperature data.

Also disclosed herein is a system of training a machine learning model to predict the probability of impairment comprising one or more processors which receive a time series of images of a subject's eye(s) labeled with the vital signs, and the psychopharmacologic test parameters associated with the data. A system of training a deep learning model to optimize the machine learning model may comprise one or more processors which receive the labeled time series of images and toxicological data.

In addition, disclosed herein is a method for generating and optimizing the processor comprising training, by one or more devices, an impairment model based on toxicologic and neurological data. The training may involve: initializing, by one or more processors, an impairment model with parameters obtained from known controls, where the deviation from normal to impaired data is known training, by one or more processors, the impairment model with adversarial normal vs impaired; training, by one or more processors, an impairment model based on a combination of published reference data, controlled subject studies, and live data from real-world users of the described system showing the progression from normal to impaired; analyzing, by one or more processors, a range of probable impairment based on each normal-to-impaired combination (e.g., Toxicologic data, neurological data) generating, by one or more processors, a weighted score of impairment based on where in the range of probable impairment the data falls; creating, by one or more processors, a summary of plots and weighed scores based on generated data; training, by one or more processors, to receive the new scores and plots and minimize the error between predicted impairment and live impairment; sending, by one or more processors, the assessment for the selected results.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

What is claimed is:

1. A method for collecting and interpreting data for determining impairment in a subject, said method comprising:
(a) measuring the state of the subject's autonomic nervous system by detecting changes in both pupillometric and vital sign parameters in response to programmed stimuli;
(b) measuring the concentration of metabolite(s) of an intoxicant and/or endogenous molecule(s) in a sample matrix that may be altered by the presence of the intoxicant metabolite(s); and
(c) analyzing the collected data by one or more instruments to determine the probability of impairment.

2. The method of claim 1 wherein the method, including steps (a), (b) and (c), is performed at the location of data collection.

3. The method of claim 1, wherein step (a) comprises determining the neurological state of the subject with an eye scanning instrument; and step (b) comprises conducting a live cell assay with a disposable cartridge.

4. The method of claim 3 further comprising:
providing the cartridge with the capacity to intake, process and/or interact with the sample matrixusing one or more eukaryotic cell-based biosensors.

5. The method of claim 1 further comprising interacting the sample matrix with a biosensing assay and transmitting optical data generated by the biosensing assay to a detector.

6. The method of claim 1 wherein a disposable cartridge accepts, filters, optionally stores and prepares the sample matrix for interaction with a biosensing assay housed in a microfluidic or millifluidic chip, and wherein:
a sample collection tool gathers the biological matrix of interest and, in the case of saliva, pre-filters larger particles from the sample; and
an on-chip mixer interacts the sample with a buffer solution stored on the chip.

7. The method of claim 1 further comprising;
automatically measuring one or more of the following pupillometric parameters with an eye scanner: lack of convergence, pupillary hippus, rebound dilation, vertical gaze nystagmus, and horizontal gaze nystagmus, by:
directing a beam of light in the VIS-NIR wavelength range from the eye scanner to a subject's eye;
detecting said parameters with a camera in the eye scanner; and
shielding the subject's eyes and internal components of the scanner from external light.

8. The method of claim 1 further comprising:
running test sequences of visible or auditory stimuli and gathering data on how a subject's vital signs respond, said vital signs comprising one or more of heart rate, respiratory rate, blood pressure, body temperature, pupillary size, and pupillary position.

9. A method of generating assays specific for the detection of an analyte or multiple analytes, utilizing one or more of the following processes:
(a) determining concentration of a target analyte in a sample matrix by comparing mutants of a receptor having different binding affinities for that target analyte;
(b) determining concentration of a target analyte concentration by comparing a signal generated by two or more biosensors expressing distinct different receptors for the same analyte.

10. The method of claim 9 wherein process (a) comprises generating the mutants through directed evolution of the receptor in the presence of the target analyte, and screening for variance in the binding affinities.

11. The method of claim 9 wherein process (a) comprises experimentally determining receptor binding domains, and inducing mutagenesis through polymerase chain reaction in the receptor binding domains to achieve enhanced variance in affinity.

12. The method of claim 9 wherein the receptor comprises at least one of: CB1, CB2, GPR18, and GPR55

13. A system for performing the methods described herein.

14. Any and all features of novelty described, referred to, exemplified, or shown herein.
